(19) United States
(12) Patent Application Publication
(10) Pub. No.: US 2025/0195204 A1
(43) Pub. Date: Jun. 19, 2025

(54) STENT ASSEMBLIES AND METHOD OF MANUFACTURING

(71) Applicant: MEDIBRANE LTD, Rosh Haayin (IL)

(72) Inventors: Elad EINAV, Salit (IL); Amir KRAITZER, Herzliya (IL)

(21) Appl. No.: 18/966,257

(22) Filed: Dec. 3, 2024

Related U.S. Application Data
(63) Continuation-in-part of application No. PCT/IB2023/055698, filed on Jun. 2, 2023.
(60) Provisional application No. 63/348,535, filed on Jun. 3, 2022.
(60) Provisional application No. 63/606,154, filed on Dec. 5, 2023.

(51) Int. Cl. A61F 2/07

(52) U.S. Cl. CPC A61F 2/07; A61F 2002/072; A61F 2220/0025; A61F 2230/0004; A61F 2230/0082; A61F 2240/001

(57) ABSTRACT

A stent assembly comprises a stent formed by a network of strut-segments and comprising an internal surface and an external surface, each strut-segment having a transverse cross-section defining a respective shape, a fabric material covering a given part of the stent on a first surface selected from the internal surface and the external surface, and a polymer binder. The covering by the fabric material is such that the fabric material is bonded to a respective transverse portion of a perimeter of each strut-segment within the given part by the polymer binder, and the respective transverse portion comprises at least 40% of the respective perimeter of each strut-segment having a transverse cross-section defining a rounded shape, and at least 60% of the respective perimeter of each strut-segment having a transverse cross-section defining a prismatic shape.

### CROSS-REFERENCE TO RELATED APPLICATIONS

This patent application is continuation-in-part (CIP) of PCT/IB2023/055698, filed on Jun. 2, 2023, which is incorporated herein by reference in its entirety. This patent application claims the benefit of U.S. Provisional Patent Application No. 63/606,154, filed on Dec. 5, 2023, which is incorporated herein by reference in its entirety. PCT/IB2023/055698 claims the benefit of U.S. Provisional Patent Application No. 63/348,535, filed on Jun. 3, 2022, which is incorporated herein by reference in its entirety. Each of the following patent applications are incorporated by reference in their entirety: (i) U.S. Provisional Patent Application No. 63/046,746 filed on Jul. 1, 2020; (ii) U.S. patent application Ser. No. 17/365,000, filed on Jul. 1, 2021, and published on Jan. 27, 2022, as US20220023027A1; and (iii) U.S. patent application Ser. No. 16/508,990, filed on Jul. 11, 2019, and published on Jan. 16, 2020, as US20200015987A1.

### FIELD OF THE INVENTION

The present invention relates to medical stent devices and assemblies comprising metal stents and covering materials (including fabrics) attached thereto, along with methods for their manufacture and assembly. In particular, the present invention is suitable for use in medical applications as a sutureless stent graft.

### BACKGROUND

A stent is a metal or polymer tube inserted into the lumen of an anatomic vessel or duct to keep the passageway open. For example, stents may be used in the vascular system, urogenital tract and bile duct, as well as in a variety of other applications in the body. Endovascular stents have become widely used for the treatment of stenosis, strictures, and aneurysms in various blood vessels. These devices are implanted within the vessel to open and/or reinforce collapsing or partially occluded sections of the vessel.

Stents are generally open ended and are radially expandable between a generally unexpanded insertion diameter and an expanded implantation diameter which is greater than the unexpanded insertion diameter. Stents are often flexible in configuration, which allows them to be inserted through and conform to tortuous pathways in the blood vessel. The stent is generally inserted in a radially compressed state and expanded either through a self-expanding mechanism, or through the use of balloon catheters.

It is also known to combine a stent and a graft to form a composite medical device. Grafts are tubular devices which may be formed of a variety of material, including textile and non-textile fabric materials and other covering materials. Such a composite medical device provides additional support for blood flow through weakened sections of a blood vessel. In endovascular applications, the use of a stent/graft combination is becoming increasingly important because the combination not only effectively allows the passage of blood therethrough, but also ensures the implant will remain open and stable.

Existing stent grafts with full bonding can require high radial forces to compress the stent for insertion into a catheter, and this can be exacerbated by resistance forces, for example, to longitudinal extension when the stent is compressed.

In addition, stent grafts with full circumferential bonding can experience non-optimal or uncontrolled wrinkling of the graft fabric when the stent is compressed and/or have a higher likelihood of kinking or twisting that can ‘choke’ the lumen of the stent-supported vessel. In addition, stent assemblies having fabric coverings attached when the stent is in an unconstrained, e.g., expanded, state can exhibit excessive fabric wrinkling when constrained to a reduced-diameter state.

### SUMMARY

According to embodiments of the invention, a stent assembly comprises: (a) a stent formed by a network of strut-segments and comprising an internal surface and an external surface, each strut-segment having a transverse cross-section defining a respective shape; (b) a fabric material covering a given part of the stent on a first surface selected from the internal surface and the external surface; and (c) a polymer binder. The covering by the fabric material is such that the fabric material is bonded to a respective transverse portion of a perimeter of each strut-segment within the given part by the polymer binder. The respective transverse portion comprises at least 30%, or at least 35%, or at least 40%, or at least 45%, or at least 50%, of the respective perimeter of each strut-segment having a transverse cross-section defining a rounded shape, and at least 55%, or at least 60%, or at least 65%, or at least 70%, of the respective perimeter of each strut-segment having a transverse cross-section defining a prismatic shape.

In some embodiments, it can be that (i) the fabric material additionally covers the given part on a second surface, such that both the internal surface and the external surface of the given part are covered, and/or (ii) the respective transverse portion comprises at least 70%, or at least 75%, or at least 80%, or at least 85%, or at least 90%, of the respective perimeter of each strut-segment having a transverse cross-section defining a rounded shape, and at least 80%, or at least 85%, or at least 90%, or at least 95%, of the respective perimeter of each strut-segment having a transverse cross-section defining a prismatic shape.

In some embodiments, it can be that each strut-segment within the given area can have a transverse cross-section defining a rounded shape, and/or that the respective transverse portion comprises at least 40% of the respective perimeter of each strut-segment. In some embodiments, it can be that each strut-segment within the given area has a transverse cross-section defining a prismatic shape, and/or that the respective transverse portion comprises at least 60% of the respective periphery of each strut-segment. In some embodiments, it can be that each strut-segment within the given area has a transverse cross-section defining a rounded shape, and/or that the respective transverse portion comprises at least 80% of the respective perimeter of each strut-segment. In some embodiments, it can be that each strut-segment within the given area has a transverse cross-section defining a prismatic shape, and/or that the respective transverse portion comprises at least 90% of the respective perimeter of each strut-segment.

In some embodiments, at least one respective transverse portion can be discontinuous. In some embodiments, all of the respective transverse portions can be discontinuous.

In some embodiments, the stent assembly can additionally comprise a coupling agent mediating between the polymer binder and the respective transverse portions. In some embodiments, the coupling agent can be effective to create a covalent bond between the polymer binder and the respective transverse portions.

In some embodiments, the stent assembly can additionally comprise a coupling agent mediating between the polymer binder and the fabric material. In some embodiments, the coupling agent can be effective to create a covalent bond between the polymer binder and the fabric material.

A method is disclosed, according to embodiments, for assembling a stent assembly. The method comprises: (a) providing a stent that comprises an internal surface and an external surface, and that is formed by a network of strut-segments, each strut-segment having a transverse cross-section defining a respective shape; (b) coating, with a polymer binder, a respective transverse portion of a perimeter of each strut-segment within a given part of the stent on a first surface selected from the internal surface and the external surface, wherein the coated respective transverse portion comprises at least 30%, or at least 35%, or at least 40%, or at least 45%, or at least 50%, of the respective perimeter of each strut-segment having a transverse cross-section defining a rounded shape, and at least 55%, or at least 60%, or at least 65%, or at least 70%, of the respective perimeter of each strut-segment having a transverse cross-section defining a prismatic shape; and (c) engaging a fabric material with the coated respective transverse portions so as to bond the fabric material thereto and thereby cover the given part of the stent on the first surface.

In some embodiments, the method can additionally comprise: (i) further coating, with the polymer binder, the respective transverse portion of the perimeter of each strut-segment within the given part of the stent on a second surface, wherein the respective transverse portion comprises at least 70%, or at least 75%, or at least 80%, or at least 85%, or at least 90%, of the respective perimeter of each strut-segment having a transverse cross-section defining a rounded shape, and at least 80%, or at least 85%, or at least 90%, or at least 95%, of the respective perimeter of each strut-segment having a transverse cross-section defining a prismatic shape, and/or (ii) additionally engaging a fabric material with the further-coated respective transverse portions so as to bond the fabric material thereto and thereby additionally cover the given part of the stent on said second surface.

In some embodiments, it can be that each strut-segment within the given area can have a transverse cross-section defining a rounded shape, and/or that the respective transverse portion comprises at least 40% of the respective perimeter of each strut-segment. In some embodiments, it can be that each strut-segment within the given area has a transverse cross-section defining a prismatic shape, and/or that the respective transverse portion comprises at least 60% of the respective periphery of each strut-segment. In some embodiments, it can be that each strut-segment within the given area has a transverse cross-section defining a rounded shape, and/or that the respective transverse portion comprises at least 80% of the respective perimeter of each strut-segment. In some embodiments, it can be that each strut-segment within the given area has a transverse cross-section defining a prismatic shape, and/or that the respective transverse portion comprises at least 90% of the respective perimeter of each strut-segment.

In some embodiments, at least one respective transverse portion can be discontinuous. In some embodiments, all of the respective transverse portions can be discontinuous.

In some embodiments, the coating can include applying a coupling agent between the polymer binder and the respective transverse portions to form a covalent bond therebetween. In some embodiments, the further coating can include applying a coupling agent between the polymer binder and the respective transverse portions to form a covalent bond therebetween. In some embodiments, the engaging can include applying a coupling agent to mediate between the polymer binder and the fabric material to form a covalent bond therebetween. In some embodiments, the additionally engaging can include applying a coupling agent to mediate between the polymer binder and the fabric material to form a covalent bond therebetween.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIGS. 1A and 1B illustrate a stent characterized by n-sided cells, according to embodiments of the present invention.

FIGS. 2A and 2B illustrate a stent characterized by undulating rings, according to embodiments of the present invention.

FIGS. 3 and 4 illustrate stent designs with different types of intersection locations, according to embodiments of the present invention.

FIG. 5 shows a perspective view of a stent coated with a polymer binder, according to embodiments of the present invention.

FIG. 6 shows a perspective view of a stent assembly according to embodiments of the present invention.

FIGS. 7A and 7B are schematic cross-sectional illustrations of bonding a fabric to a stent by having a polymer binder enter pores in the fabric, according to embodiments of the present invention;

FIG. 8 shows a flowchart of a method for attaching a fabric material to a stent comprising a metal alloy, according to embodiments of the present invention.

FIGS. 9A and 9B illustrate a stent assembly characterized by stent-assembly segments, according to embodiments of the present invention.

FIGS. 10A and 10B are schematic illustrations of intersecting struts coated with a polymer binder, according to embodiments of the present invention.

FIG. 11 shows a flowchart of a method for producing a radially compressible stent assembly comprising longitudinally displaced stent-assembly segments with different respective radial strengths, according to embodiments of the present invention.

FIGS. 12A and 12B are schematic cross-sectional illustrations of binder wings provided to facilitate bonding of a fabric to a stent, according to embodiments of the present invention.

FIG. 13 shows a flowchart of a method for producing a stent assembly comprising a metal stent formed by a network of struts and having an internal major surface and an external major surface, according to embodiments of the present invention.

FIG. 14 illustrates the painting of a binder onto a stent with fabric engaged therewith, according to embodiments of the present invention.

FIGS. 15A and 15B illustrate examples of stent designs in which a fabric material can be attached to portions of stents, according to embodiments of the present invention.

FIGS. 16A-16E illustrate examples of different shapes and designs of stents incorporating embodiments of the present invention.

FIG. 17A shows, schematically, a stent with multiple binder locations disposed circumferentially around a transverse section of the stent, according to embodiments of the present invention.

FIG. 17B shows a detail of FIG. 17A.

FIG. 18 shows, schematically, a fabric graft selectively bonded to the stent of FIG. 17A, according to embodiments of the present invention.

FIG. 19 shows, schematically, a stent with multiple binder locations disposed circumferentially around a transverse section of the stent, according to embodiments of the present invention.

FIG. 20A shows, schematically, a stent with multiple binder locations disposed circumferentially around each of a plurality of transverse sections of the stent, according to embodiments of the present invention.

FIG. 20B shows a detail of FIG. 20A.

FIG. 21 shows, schematically, a stent with multiple binder locations disposed circumferentially around each of a plurality of transverse sections of the stent, according to embodiments of the present invention.

FIG. 22 shows, schematically, a stent with multiple binder locations disposed circumferentially around each of a plurality of transverse sections of the stent with a staggered pattern of selective bonding, according to embodiments of the present invention.

FIGS. 23A and 23B show, schematically, a cutaway cross-section of a selectively-bonded stent graft according to embodiments of the present invention, respectfully, before and after the application of compressive radial force.

FIG. 24 shows a graph of experimental results of measuring loading force on stents manufactured according to embodiments of the present invention as a function of selective bonding percentage.

FIG. 25 shows a flowchart of a method of attaching a fabric material to a stent formed by a network of struts using selective bonding according to embodiments of the present invention.

FIGS. 26A, 26B, 26C, and 26D show respective schematic cross-sections of various examples of struts coated with a primer, encapsulated with a polymer binder to a first thickness, and coated with the polymer binder to a second thickness, according to embodiments of the present invention.

FIGS. 27A and 27B show flowcharts of method steps for attaching a covering material to a stent formed by a network of struts, according to embodiments of the present invention.

FIGS. 28A and 28B show flowcharts of method steps for attaching a fabric material to a metal-alloy stent formed by a network of struts, according to embodiments of the present invention.

FIGS. 29A, 29B, 29C, 29D, 29E, 29F, and 29G schematically illustrate stages and/or method steps for attaching a fabric material to a metal-alloy stent formed by a network of struts, according to embodiments of the present invention.

FIG. 30A is a schematic illustration of a stent scaffold, according to embodiments of the present invention.

FIG. 30B is a detail view of FIG. 30A, showing several strut segments, according to embodiments of the present invention.

FIG. 30C shows a transverse cross-section of a strut segment, according to embodiments of the present invention.

FIG. 31 shows a stent graft with a fabric material covering both interior and exterior surfaces of the stent, with a given area of the stent identified, according to embodiments of the present invention.

FIG. 32A shows a cross-sectional view of a strut segment having a rounded shape, with fabric material bonded to a transverse portion of the perimeter of the strut segment on a single surface, according to embodiments of the present invention.

FIGS. 32B and 32C show respective cross-sectional views of a strut segment having a rounded shape, with fabric material bonded to a transverse portion of the perimeter of the strut segment on two surfaces, according to embodiments of the present invention.

FIG. 33A shows a cross-sectional view of a strut segment having a rounded shape, with fabric material bonded to a transverse portion of the perimeter of the strut segment on a single surface, according to embodiments of the present invention.

FIGS. 33B and 33C show respective cross-sectional views of a strut segment having a rounded shape, with fabric material bonded to a transverse portion of the perimeter of the strut segment on two surfaces, according to embodiments of the present invention.

FIG. 34A shows a cross-sectional view of a strut segment having a prismatic shape, with fabric material bonded to a transverse portion of the perimeter of the strut segment on a single surface, according to embodiments of the present invention.

FIGS. 34B and 34C show respective cross-sectional views of a strut segment having a prismatic shape, with fabric material bonded to a transverse portion of the perimeter of the strut segment on two surfaces, according to embodiments of the present invention.

FIG. 35A shows a cross-sectional view of a strut segment having a prismatic shape, with fabric material bonded to a transverse portion of the perimeter of the strut segment on a single surface, according to embodiments of the present invention.

FIGS. 35B and 35C show respective cross-sectional views of a strut segment having a prismatic shape, with fabric material bonded to a transverse portion of the perimeter of the strut segment on two surfaces, according to embodiments of the present invention.

FIGS. 36A and 36B illustrate respective stent scaffolds wherein struts have a prismatic cross-sectional shape, curved with the contour of the stent, according to embodiments of the present invention.

FIG. 36C is a schematic cross-sectional view of a stent scaffold wherein struts have a prismatic cross-sectional shape, curved with the contour of the stent, according to embodiments of the present invention.

FIG. 36D shows a cross-sectional view of a strut segment having a prismatic shape, curved with the contour of the stent, with fabric material bonded to a transverse portion of the perimeter of the strut segment on a single surface, according to embodiments of the present invention.

FIGS. 37A and 37B are schematic illustrations of cross-sections of exemplary fabric-covered struts of stent assemblies according to embodiments of the present invention.

FIGS. 37C and 37D are schematic illustrations of alternative structures of fabric layers, according to embodiments of the present invention.

FIG. 37E schematically illustrates an impermeable component of a fabric layer entering one or more permeable layers, according to embodiments of the present invention.

FIGS. 38 and 39 show flowcharts of methods and method steps for assembling a stent assembly, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements. Subscripted reference numbers (e.g., 10₁) or letter-modified reference numbers (e.g., 100a) are used to designate multiple separate appearances of elements in a single drawing, e.g. 10₁ is a single appearance (out of a plurality of appearances) of element 10, and 100a is a single appearance (out of a plurality of appearances) of element 100.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

The term ‘stent assembly’ as used herein means an assembly of a medical stent and a fabric cover, sleeve or attachment, attached to the struts of the stent. A stent assembly can be what is commonly called a stent graft. A stent assembly can additionally include materials used in the assembly, such as, for example, primers, coatings, binders and polymers or elastomers.

The term ‘bonding’ can be used to mean a process of joining using any one or more of: applying an adhesive binder, e.g., by painting it onto a surface of a stent strut; applying heat; and applying pressure. The bonding can be carried out by applying a binder to a strut and then engaging the fabric, or by engaging the fabric and then applying the binder. Either approach can be practiced in any of the embodiments disclosed herein. In some cases, the bonding or adhesion is chemical in nature, e.g., when the binder and fabric comprise materials which form adhesive chemical bonds therebetween. In some cases, the bonding or adhesion is mechanical in nature, e.g., when a mechanical interlock is achieved, such as when a binder enters the pores of a porous fabric. In some cases, the bonding or adhesion can achieve a combination of chemical and mechanical adhesion. All of these cases can be implemented in accordance with any of the embodiments disclosed herein.

The terms ‘covering material’ and ‘fabric material’ ('fabric) are used interchangeably throughout this disclosure and in the claims appended thereto except where fabric is modified by ‘woven’ or ‘porous’ and the like'. In another words, a covering material, or, equivalently, can include, and not exhaustively a fabric material, whether woven or not, or a non-fabric material, such as an elastomeric material, or any permeable or impermeable deployed as a covering material (or ‘graft’) for a stent.

In some embodiments, a stent assembly comprises a stent and a porous fabric material. In other embodiments, a stent assembly comprises a stent and a non-porous fabric, which can be liquid-impermeable. The fabric/covering material can be in the form of a sheet or a sleeve, e.g., a cylinder.

### First Discussion of Embodiments

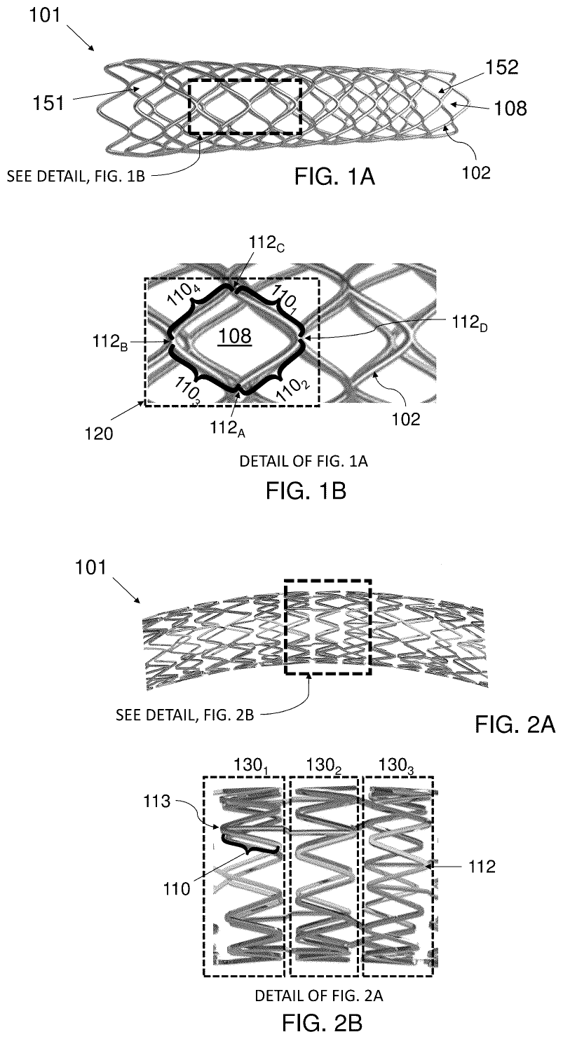

A first example of a stent is shown in FIGS. 1A and 1B. The stent 101 is formed by a network of struts 102. The stent 101 has an internal surface 151 and an external surface 152. As seen, each of the surfaces 151, 152 comprises surfaces of struts 102, and open spaces 108 between the struts 102. The network of struts 102 can comprise a plurality of strut segments 110 defined by intersection locations 112. The area of either surface 151, 152 of the stent 102 can be thought of as comprising a plurality of stent-area portions.

In the embodiment illustrated in FIGS. 1A and 1B, the stent-area portions comprise 4-sided cells 120 each comprising 4 strut segments (e.g., 110₁, 110₂, 110₃, 110₄) defined by 4 intersection locations (e.g., 112_{A}, 112_{B}, 112_{C}, 112_{D}). More generally, the stent-area portions, in embodiments, can comprise n-sided cells each comprising n strut segments defined by n intersection locations, where n is an integer equal to at least 3 and at most 6. In various embodiments, the cells can have either regular polygon shapes or irregular shapes. Strut segments 110 can be mostly straight or curved in accordance with a stent design. A stent 101 can have at least 1, at least 10, at least 20, at least 50, at least 100 or more such n-sided cells. The n-sided cells can make up one or more regions of the surfaces 151, 152 of a stent 101, or can account for the entire stent surface.

Referring now to FIGS. 2A and 2B, another example of stent design is shown where the network of struts 102 is characterized by undulating rings 130 of strut segments 102 defined by bends 113. Bends 113 are another form of intersection locations like the intersection locations 112 of FIG. 1B, in that they mark where strut segments 102 intersect. As mentioned earlier, the stent 101 can be thought of as comprising a plurality of stent-area portions, in the embodiment illustrated in FIGS. 2A and 2B, the stent-area portions comprise undulating rings 130 (e.g., 130₁, 130₂, 130₃). A stent 101 can have at least 1, at least 5, at least 10, at least 20, or more such undulating rings 130. The undulating rings 130 can make up one or more regions of the surfaces 151, 152 of a stent 101, or can account for the entire stent surface.

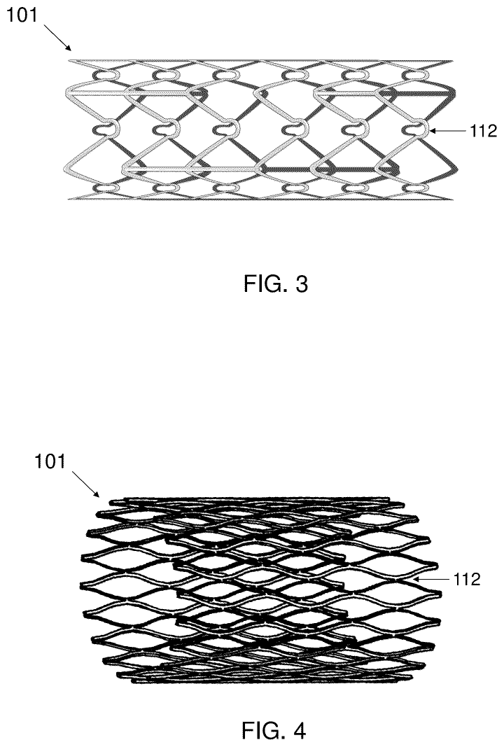

FIGS. 3 and 4 illustrate additional examples of stents 101 characterized by n-sided cells 120, wherein the intersection locations 112 are different from those shown, for example, in FIGS. 1A and 1B. In FIG. 3, the intersection locations 112 comprise overlapping hooks. In FIG. 4, the 4-sided cells 120 are laterally compressed and the constituent strut segments are somewhat curved; the corresponding intersection locations 112 are located where two such adjacent ‘curves’ touch each other. In spite of having a different form than the earlier examples of n-sided cells 120 and intersection locations 112, the actual design is not material to the invention, and any suitable strut design can be used. Similarly, the undulating rings 130 of FIGS. 2A and 2B can be, in embodiments, less regular and/or can have more complex shapes.

FIG. 5 shows a view of a stent 101 coated with a polymer to form a polymer binder layer 104, according to an embodiment. Generally, the medical stent or stent 101 is a tiny tube, comprising a plurality or network of struts 102 configured to form a mesh like structure. The width of the strut 102 is typically between 1-2 mm, although it can be narrower or wider according to specific stent designs. The term ‘width’ as used herein when applied to the strut of a stent refers to a measure of the strut's ‘footprint’ on the surface of the stent. According to the present invention, surfaces of the struts 102 of the stent 101 can be coated with a polymer binder. In embodiments, the binder layer 104 can comprise a elastomer, e.g., a thermoplastic elastomer, chosen for its thermoplastic and elastomeric properties. Examples of suitable thermoplastic elastomers include styrenic block copolymers, thermoplastic polyolefinelastomers, thermoplastic vulcanizates, thermoplastic polyurethanes, thermoplastic copolyesters, and thermoplastic polyamides.

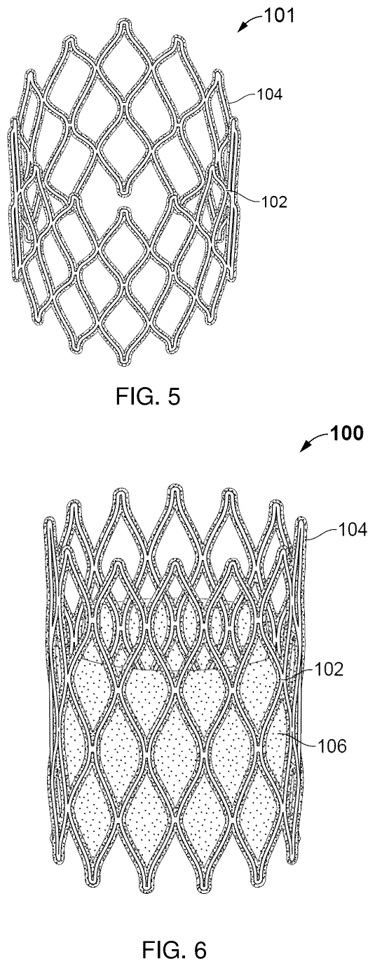

We now refer to FIG. 6 which shows a stent assembly 101 comprising a stent 101 with struts 102 coated with a polymer binder 104 and partly covered by a fabric material 106. In one embodiment, the fabric material 106 is attached to struts 102 of by means of a solvent bonding technique. In another embodiment, the fabric material 106 is attached to the struts 102 by means of heat and pressure application. In some embodiments, the fabric material 106 is fabricated from any desired fiber material used in the industry for stent sleeves, covers and grafts, such as, but not limited to, electro spun fibers, expanded polytetrafluoroethylene (EPTFE), Polyethylene terephthalate (PET or PETE) or thermoplastic polyurethane (TPU) film. The fabric material 106 can be manufactured by means of any known manufacturing technology, but not limited to woven, non-woven, knitting or electrospinning techniques.

In some embodiments, a porous fabric material can be deployed to cover at least a portion of at least one of the internal surface 151 and the external surface 152, and bonded to the metal stent 101 by a polymer binder 104 that mediates between the struts 102 and the fabric material 106 to bind therebetween.

As seen in FIG. 6, in some embodiments, the fabric material 106 covers, at least 60%, by length, of the combined lengths of the struts 102 of the network within a first surface region of the stent, and therefore at least 60%, by length, of the combined lengths of the struts 102 of the network within a first surface region of the stent is coated with the polymer binder 104. In other embodiments, at least 70%, at least 80%, or least 90% of the combined lengths of the struts 102 can be coated with the polymer binder 104.

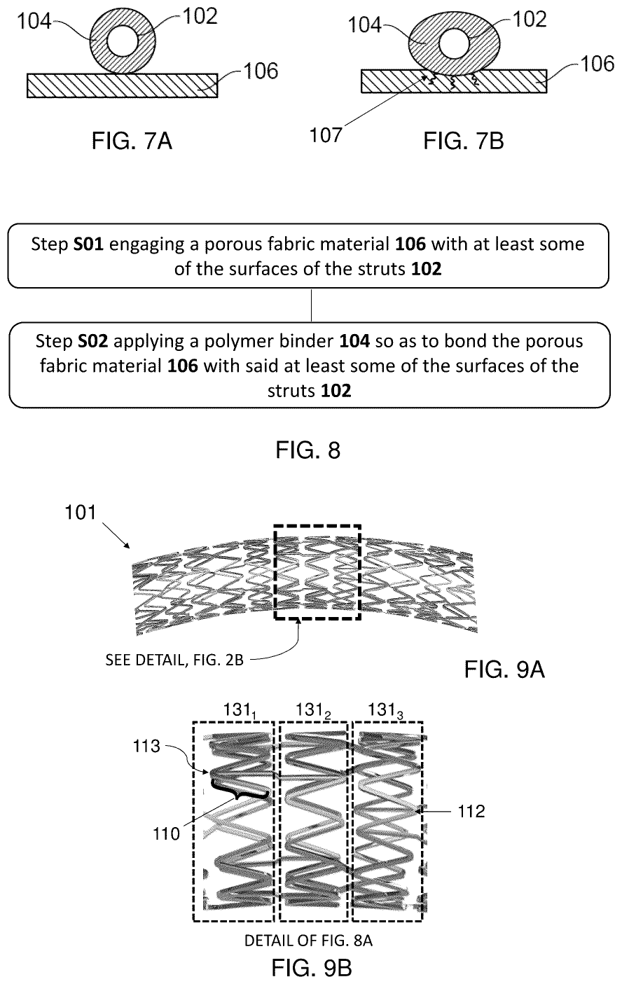

As is illustrated schematically in FIGS. 7A and 7B, it can be desirable for the polymer binder 104 to enter pores 107 in fabric 106 as a way of making the binding/bonding between fabric 106 and more effective. Returning to FIG. 6, the polymer binder 104 is applied (or expands/flows/is squeezed by pressure) close to the struts 102 so as to enter pores 107 in the fabric 106 and is not to be found far from the struts 102. In embodiments, for a given region (or multiple regions) comprising one or more stent-area portions, at least 70% or at least 80% of the area of the fabric material 106 that is “close to struts 102” is rendered non-porous (meaning at least 90% non-porous) by a presence of the polymer binder 104 within pores 107 of the fabric material 106. “Close to struts 102” can be interpreted as with 0.5 mm, or within 1.0 mm, or with 2.0 mm, where the distance is measured laterally from lateral edges of the struts 102. Similarly, in those region(s), at least 70% or at least 80% of the area the fabric 106 in of portions of the fabric material that is “far from struts 102” is characterized by pores 107 that are free (meaning at least 90% free) of the polymer binder 104. “Far from struts 102” can be interpreted as at least 1 mm, at least 2 mm, or at least 3 mm displaced laterally from the lateral edges of struts 102.

It should be noted that any of the foregoing criteria (e.g., with respect to at least 70% or at least 80% of the area of the fabric material 106 that is “close to struts 102” being rendered non-porous, or with respect to at least 70% or at least 80% of the area the fabric 106 in of portions of the fabric material that is “far from struts 102” being characterized by pores 107 that are free of the polymer binder 104) can be applied globally for all stent-areas (e.g., n-sided cells 110 and/or undulating rings 130) in a region of the stent or even over the entire stent, but can also be applied at the individual stent-area (n-sided cell 110 and/or undulating ring 130) level, such that in some embodiments the criteria are applied within each individual one of the stent-areas.

According to some embodiments, the thickness of the polymer binder 104 is not less than 1 micron and not greater than 70 microns, although ins some embodiments the binder layer can be thicker, e.g., 80-100 microns. In some embodiments, the thickness of the polymer binder 104 is not less than 5 microns and not greater than 40 microns. In embodiments, typical strut thicknesses are in the range 100-200 microns, although struts can be more or less thick. In embodiments, a ratio of strut thickness to polymer binder thickness is at least 1.5, or at least 2.0, or at least 2.5, or at least 3.0.

In embodiments, the struts 102 are encapsulated with the polymer 104 and the fabric material 106 is attached thereto by the application of heat and pressure. In some embodiments, the fabric 106 is engaged (i.e., brought in contact) with the bare metal strut 102 and the polymer binder 104 is then applied so as to bind therebetween. In some embodiments, the polymer 104 is melted and flows into a plurality of pores 107 characteristics of the fabric 106 enabling a strong bond between the fabric material 106 and struts 102. In some embodiments, the melting point of fabric 106 is greater than the melting point of the polymer by at least 10° C.

The flowchart in FIG. 8 illustrates a method for attaching a fabric material 106 to a stent 101 comprising a metal alloy, where the stent 101 is formed by a network of struts 102 and having an external surface 151 and an internal surface 152. The method comprises:

Step S01 engaging a porous fabric material 106 with at least some of the surfaces of the struts 102; and

Step S02 applying a polymer binder 104 so as to bond the porous fabric material 106 with said at least some of the surfaces of the struts 102. The applying is such that: (i) at least 90%, by length, of the combined lengths of the struts of the network within a first surface region of the stent, are bonded to the porous fabric material by polymer binder, (ii) at least 70%, by area, of the fabric material disposed (A) within each one of one or more stent-area portions within a second surface region of the stent and (B) no more than 2 mm from a nearest respective strut, is rendered non-porous by a presence of the polymer binder within pores of the fabric material, (iii) at least 70%, by area, of portions of the fabric material (A) disposed within each one of said one or more stent-area portions and within said second surface region and (B) distanced at least 3 mm from a nearest respective strut, is characterized by pores that are free of the polymer binder, and (iv) the thickness of the polymer binder is not less than 1 micron and not greater than 70 microns.

In some embodiments, the fabric 106 can receive surface treatment, additionally or alternatively to the surface treatment of struts 102, so as to improve the bonding of the fabric with the struts.

### Second Discussion of Embodiments

Precise application of polymer binder to struts can be desirable because it facilitates control of physical parameters of a stent or stent assembly, such as, for example, radial strength (also called radial resistance). In embodiments, radial strength (a measure of stiffness of a segment of a stent can be a function of the lateral thickness of the polymer binder coating at or adjacent to intersection locations of struts, assuming all else (material, strut thickness) is held constant.

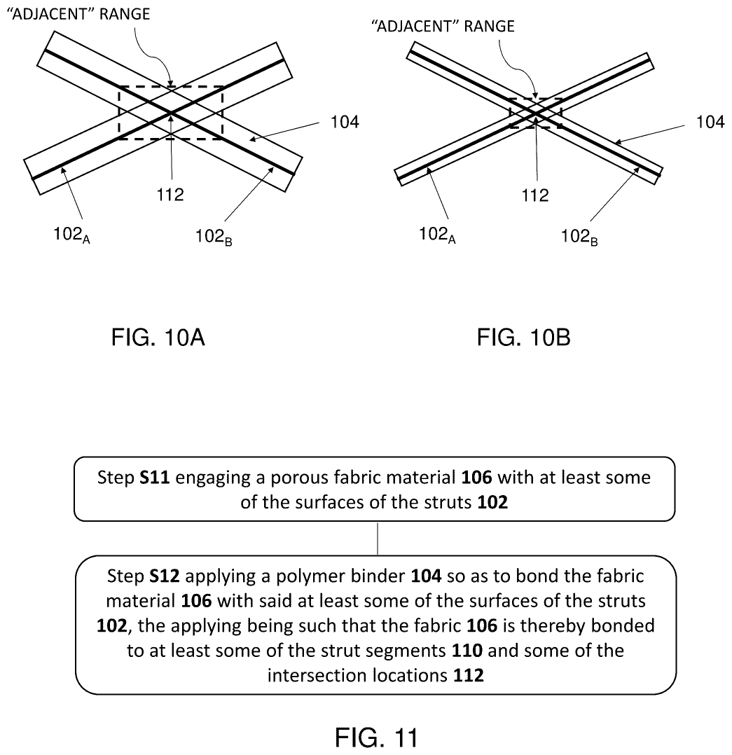

With precise control of radial strength, it is possible to deploy a stent with different radial strengths in different segments. Referring now to FIGS. 9A and 9B (similar to FIGS. 2A and 2B, except that in FIGS. 9A and 9B the undulating rings 130 of the earlier figures are used to embody stent segments 131), in an embodiment, stent segment 131₂ can have a radial strength that is higher (e.g., at least 20% higher, at least 50% higher, or at least 80% higher) than stent segments 131₁ and 131₃, depending on the thickness of a polymer binding (not shown in FIGS. 2A and 2B) applied at or adjacent to intersection locations 112. Such segments are obviously not limited to stents characterized by the undulating rings of FIGS. 2A and 2B and can alternatively or additionally include stent surface regions characterized by n-sided cells. The term “adjacent” is used to mean within an “adjacent range” where the polymer binder 104 of one strut 102 intersects with the polymer binder 104 of another strut 102 at an intersection location 112, as illustrated schematically FIGS. 10A and 10B.

In FIGS. 10A and 10B, it can be seen that the polymer binder 104 can be applied with different lateral thicknesses depending, inter alia, on the desired radial strength, i.e., although the figures accompanying this specification are not drawn to scale, the thickness of polymer binder 104 in FIG. 10A has been deliberately and exaggeratedly shown to be much thicker than in FIG. 10B. It will be obvious to the skilled artisan that the illustration of FIGS. 10A and 10B is equally applicable to a stent surface region where the intersection locations are characterized by bends (like those in FIGS. 2A and 2B). The term ‘lateral thickness’ as used with respect to the polymer binder refers to the dimension of the binder on the strut as measured in a lateral direction, i.e., on and along the surface of the stent and is not a ‘thickness’ which would be measured through the surface of the stent inwards our outwards.

In embodiments, a radially compressible covered stent assembly 100 comprises first and second stent-assembly segments 131 displaced from one another longitudinally, the stent assembly 100 has an external surface 152 and an internal surface 151, and the stent assembly comprises: (a) a radially compressible stent 101 formed by a network of struts 102, the network having a plurality of intersection locations 112 at which intersections and/or bends define strut segments 110, each strut segment 110 having respective outward-facing and inward facing-surfaces which correspond to the external and internal surfaces of the stent assembly 152, 151, and two respective laterally-facing surfaces; (b) a polymer binder 104 applied to the struts 102 so as to at least partially coat at least some of the strut segments 110 and at least some of the intersection locations 112; and a fabric 106 covering at least a portion of at least one of the internal surface 151 and the external surface 152 so as to be in contact with the polymer binder 104, where at least 80% of said contact is characterized by the polymer binder 104 forming a bond between the fabric 106 and a strut segment 110 or intersection location 112 at the respective point of contact. First and second stent-assembly segments 131 are at least partially coated with the polymer binder 104, the polymer binder 104 having respective first and second thicknesses on one or both laterally-facing surfaces of at least some respective strut segments 110 at or adjacent to respective intersection locations 112, said first and second thicknesses being different from each other. The radial strength of the first stent-assembly segment 131 is greater than the radial strength of the second stent-assembly segment 131. In some embodiments, the thickness are different from each other by at least 10% and the radial strength of the segments 131 differs by at least 20%.

These embodiments can be beneficially combined with any of the other embodiments disclosed herein, e.g., with respect to the network of struts being characterized by n-sided cells (as in FIGS. 1A and 1B), with respect to the network of struts being characterized by undulating rings (as in FIGS. 2A and 2B), or with respect to the stated ranges of thickness of the polymer binder. In embodiments, the network of struts 102 can comprise a plurality of strut segments 110 defined by intersection locations 112, where an n-sided cell comprises n strut segments 110 defined by n intersection locations 112, where n is an integer equal to at least 3 and at most 6, and at least 70% or at least 80% of the surface area of the metal stent is characterized by n-sided cells. In embodiments, the network of struts 102 includes a plurality of undulating rings 130 of defined by bends, and at least 70% of the surface area or at least 80% of the metal stent 101 is characterized by undulating rings 130. In embodiments, the thickness of the polymer binder 104 is not less than 1 micron and not greater than 70 microns.

The flowchart in FIG. 11 illustrates a method for producing a radially compressible stent assembly 100 comprising longitudinally displaced stent-assembly segments 131 with different respective radial strengths, the stent assembly 100 comprising a radially compressible stent 101 formed by a network of struts 102, the network having a plurality of intersection locations 112 at which intersections and/or bends define strut segments 110, each strut segment 110 having respective outward-facing and inward facing-surfaces which correspond to the external and internal surfaces 152, 151 of the stent assembly, and two respective laterally-facing surfaces. The method comprises:

Step S11 engaging a fabric material 106 with at least some of the surfaces of the struts 102; and

Step S12 applying a polymer binder 104 so as to bond the fabric material 106 with said at least some of the surfaces of the struts 102, the applying being such that the fabric 106 is thereby bonded to at least some of the strut segments 102 and some of the intersection locations 112. The applying includes applying a first coating thickness on one or both laterally-facing surfaces of at least some respective strut segments 110 at or adjacent to respective intersection locations 112 of a first stent-assembly segment 131 and applying a second coating thickness on one or both laterally-facing surfaces of at least some respective strut segments 110 at or adjacent to respective intersection locations 112 of a second stent-assembly segment 131; the respective stent-assembly segments 131 have different radial strengths that are a function of the coating thicknesses.

### Third Discussion of Embodiments

According to embodiments, it can be desirable to bond a non-porous fabric material to a stent, i.e., without the benefit of binder filling pores to improve the efficiency of bonding. For example, lateral ‘wings’ of binder material on either side of a stent strut can be provided so as to increase the area of binding contact between the polymer binder and the fabric. The wings are provided laterally, i.e., locally parallel to the surface of the stent without necessarily thickening the coating on the inward-facing or outward-facing surfaces of the struts.

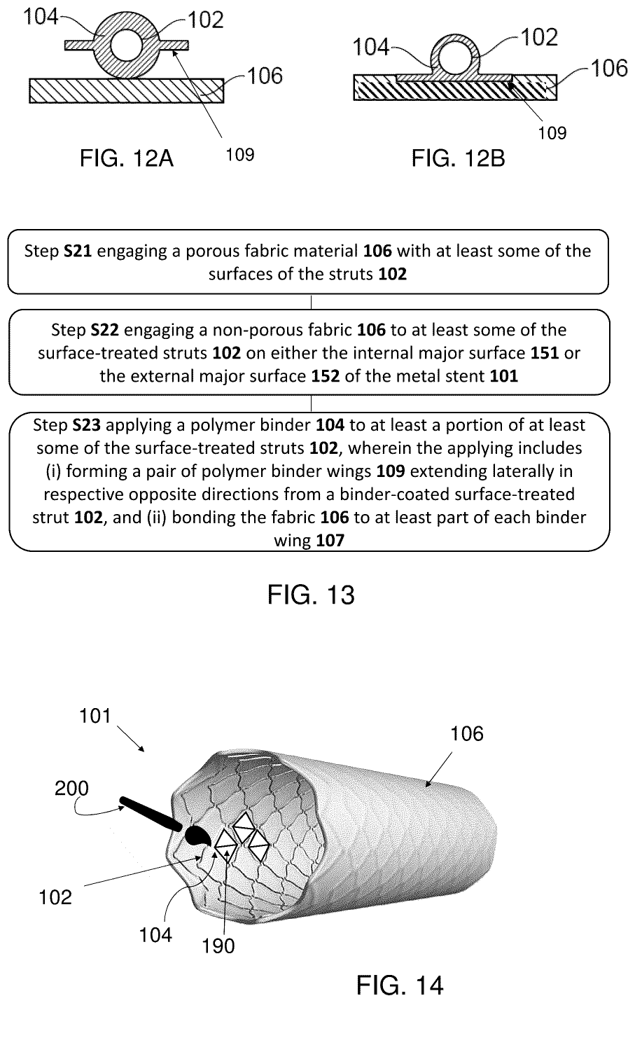

Examples of wings are shown in FIGS. 12A and 12B. In FIG. 12A, wings 109 are formed laterally from strut 102 and extent the polymer coating 104 to both sides of the strut 102. In FIG. 12B, the wings 109 are bonded with a corresponding area of the surface of the fabric 106.

In embodiments, a fabric 106 can cover all, part, or none of one of the surfaces 151, 152 of a stent 101. A coverage-value reflects to what extent a surface is covered. For example, a 100% coverage-value means that a surface is completely covered, a 50% coverage-value means that 50% of the area of a surface is covered, and a 0% coverage-value means that a surface has no fabric cover at all.

In embodiments, a stent assembly 100 comprises: a metal stent 101 formed by a network of struts 102 and having an internal major surface 151 and an external major surface 152; a polymer binder coating 104, covering at least a portion of at least some of the struts 102, and having a thickness not less than 1 micron and not greater than 70 microns (in some embodiments 5-40 microns); and a fabric 106 at least partly covering at least one of the two major surfaces 151, 152 and bonded thereto by the binder coating 104, such that a first surface (i.e., internal or external surface 151 or 152) has a coverage-value of no less than 50%, and the second surface (i.e., the other of the two surfaces 151, 152) has a coverage-value of at most 50% of the coverage-value of the first surface. In an example, a first surface has a coverage-value of over 90% and the second surface has a coverage-value of 0%. In another example, a first surface has a coverage-value of 50% and the second surface has a coverage value of 10%. Further, the binder coating 104 forms a pair of binder-coating wings 109 extending laterally in respective opposite directions from each of a plurality of the binder-coated struts 102, and the bonding of the fabric 106 to the surface of the stent 101 includes bonding the fabric 106 to at least part of each binder wing 109. In some embodiments, the coverage-value of the second surface is zero. In some embodiments, the fabric 106 can be a non-porous, liquid impermeable film.

The flowchart in FIG. 13 illustrates a method for producing a stent assembly 100 comprising a metal stent 101 formed by a network of struts 102 and having an internal major surface 151 and an external major surface 152, and (ii) a liquid-impermeable fabric 106 at least partly covering a single one of the two major surfaces 151, 152. The method comprises:

Step S21 surface-treating at least some of the surfaces of the struts 102; Surface treatment are known in the art and can be mechanical (e.g., sandblasting, creating pits or striations, etc., to increase surface area), or chemical (e.g., etching, eroding, dipping, etc.).

Step S22 engaging a non-porous fabric 106 to at least some of the surface-treated struts 102 on either the internal major surface 151 or the external major surface 152 of the metal stent 101; and

Step S23 applying a polymer binder 104 to at least a portion of at least some of the surface-treated struts 102, wherein the applying includes (i) forming a pair of polymer binder wings 109 extending laterally in respective opposite directions from a binder-coated surface-treated strut 102, and (ii) bonding the fabric 106 to at least part of each binder wing 107. In some embodiments, the applying includes painting.

In some embodiments, the fabric 106 can receive surface treatment, additionally or alternatively to the surface treatment of struts 102, so as to improve the bonding of the fabric with the struts.

FIG. 14 shows a metal stent, with a fabric material 106 engaged on the external surface of the stent. The example shown in FIG. 14 is of a stent with fabric only on the external surface, but it will obvious to the skilled practitioner that the teaching herein applies equally to a stent with fabric on the internal surface and to stents fabric on both major surfaces, i.e., internal and external. According to embodiments, a polymer binder 104 is applied to the struts 102. In some embodiments, the application is by painting the binder onto the struts, as indicated schematically by paintbrush 200. In some embodiments, sections of fabric 106 defined by cells (such as n-sided cells 110 of FIG. 1B) can be ‘masked’ using masks 190 to prevent painting the fabric 106 with binder material. Masks can be attached to each other to allow masking of a large portion (or all) of a major surface of the stent.

### General Discussion

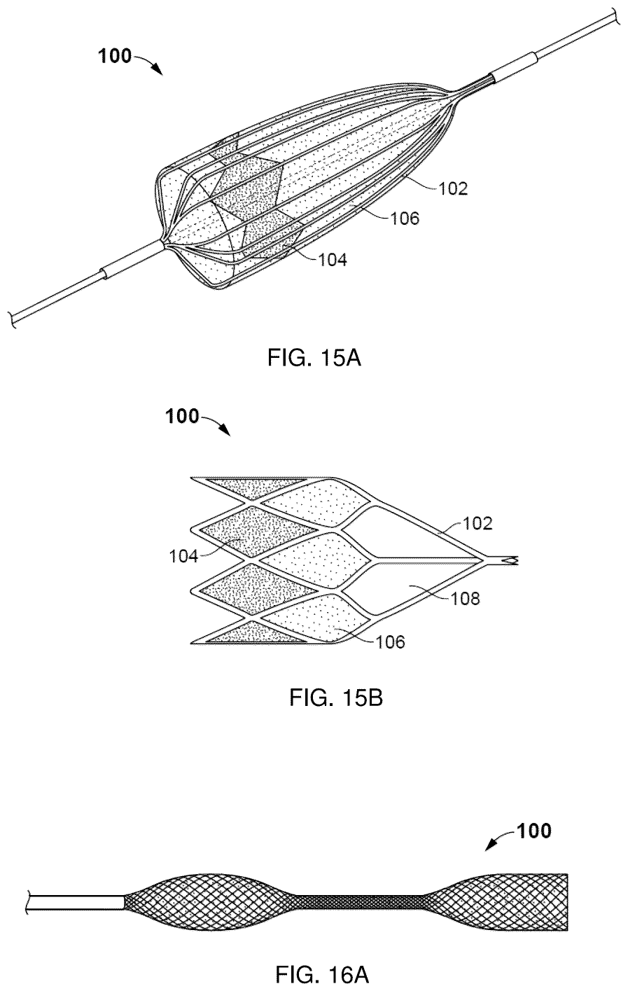

FIGS. 15A and 15B illustrate examples for attaching the fabric material 106 to various stents and expanded frames according to embodiments. In an embodiment, some portion of the stent or frame 101 could be covered by polymer film or layer 104, some portion of the stent or frame 101 could be covered by fabric material 106, and further some portion of the stent or frame 101 could be open 108, i.e., not covered with any material.

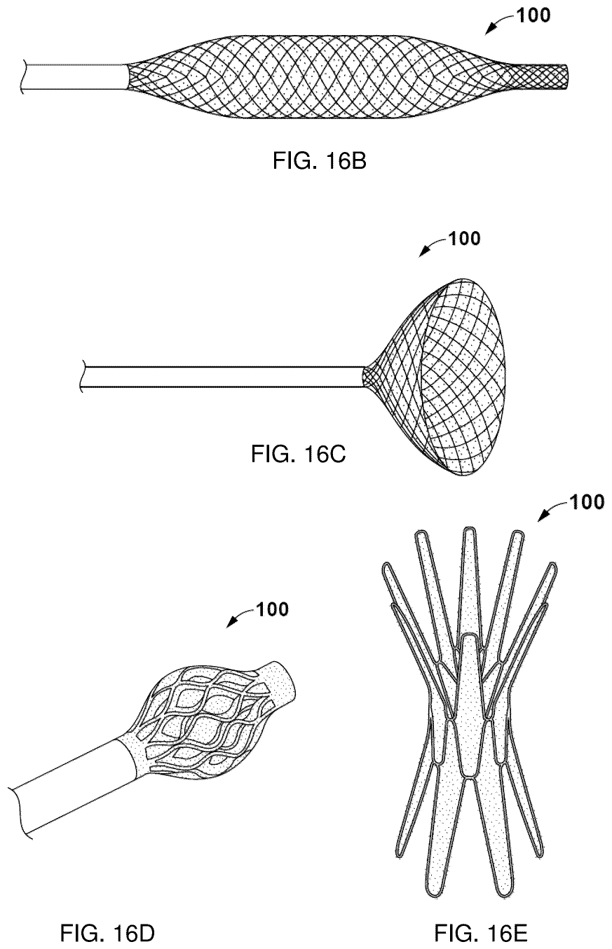

FIGS. 16A-16E illustrate examples of different shapes and designs of stent assemblies 100 incorporating embodiments of the present invention. As seen, the stent assembly 100 can be configured in any desirable shape, and is not limited to conical or cylindrical/tubular shapes.

### Selective Bonding

In embodiments, it can be desirable to selectively bond the fabric 106 to the stent 101. A fabric (i.e., any covering material) 106 can be selectively bonded to a stent 101 to form a stent graft/assembly 101. The term ‘selective bonding’ is used to describe bonding, i.e., application of a binder such as polymer-based binder 104 that is applied at selected locations on a stent 101. In a non-limiting example, selective bonding is used to reduce the radial forces required to compress a stent assembly 100, e.g., for loading into a catheter. In another non-limiting example, selective bonding is used to facilitate control of wrinkling of a fabric cover 106 around the circumference of a stent 101 when the stent 101 is compressed. In another non-limiting example, selective bonding is used to reduce the resistance force of longitudinal expansion encountered when radially compressing a stent 101 and/or the resistance forces that lead to kinking and/or twisting of a stent 101.

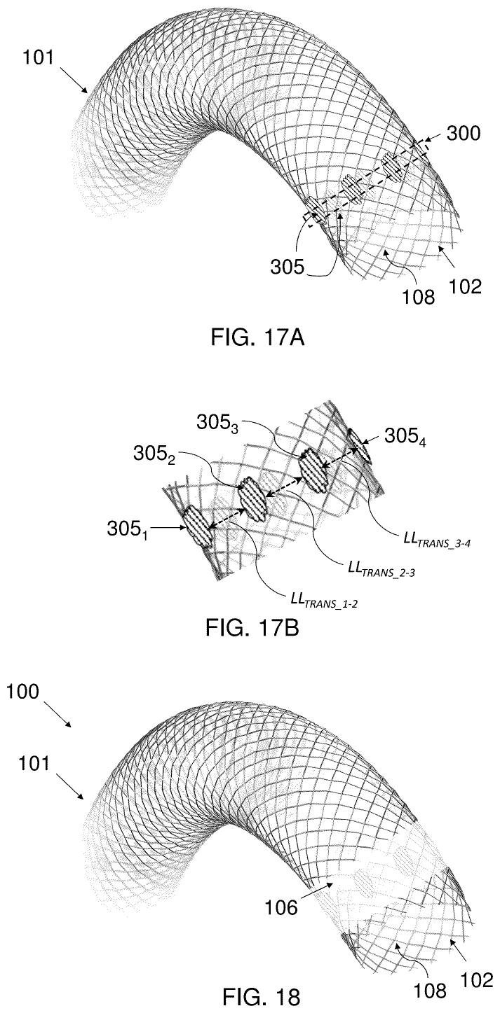

Referring now to FIGS. 17A and 17B, a ring-like transverse section 300 of a stent 101 encompasses a circumference of the stent 101. The transverse section 300 can be of any longitudinal length; for example, a transverse section can between 0.01 mm and 5 mm, or between 0.01 mm and 10 mm. The circumference of the transverse section 300 intersects, at least partially, a plurality of selectively-bonded binder locations 305. The locations 305 in FIG. 17A are spaced around the circumference of the transverse section 300 with a regular location-location spacing. As shown in the cutaway detail of FIG. 17B, the location-location spacing between any two adjacent locations 305 along the circumference of the transverse section 300 is represented by LL_{TRANS}, such that the transverse location-location spacing between locations 305₁ and 305₂ is LL_{TRANS_1-2}, the transverse location-location spacing between locations 305₂ and 305₃ is LL_{TRANS_2-3}, and the transverse location-location spacing between locations 305₁ and 305₂ is LL_{TRANS_3-4}. Regular location-location spacing means that all of the respective location-location spacings around the circumference of a given transverse section 300 are the same as each other, or within ±5% of each other, or within ±10% of each other, or within ±15% of each other, or within ±20% of each other, or within ±25% of each other, or within ±30% of each other. In other words, the transverse location-location spacings LL_{TRANS_1-2}, LL_{TRANS_2-3}, and LL_{TRANS_3-4} are all the same, or all fall within an allowed-variation range of 95%-100% of the length of the longest LL_{TRANS} of a given transverse section 300, or within a length range of 95%-100% of the length of the longest LL_{TRANS} of the transverse section 300, or within a range of 90%-100% of the length of the longest LL_{TRANS} of the transverse section 300, or within a length range of 85%-100% of the length of the longest LL_{TRANS} of the transverse section 300, or within a length range of 80%-100% of the length of the longest LL_{TRANS} of the transverse section 300, or within a length range of 75%-100% of the length of the longest LL_{TRANS} of the transverse section 300, or within a length range of 70%-100% of the length of the longest LL_{TRANS} of the transverse section 300.

The number of binder locations 305 shown in FIGS. 17A and 17B for the transverse section 300 is merely illustrative of a non-limiting example, and there can be fewer or more locations 305 on the circumference of any given transverse section 300, for example from 3 to 24, or from 4 to 18, or from 5 to 12, or from 6 to 9, all ranges inclusive. A skilled artisan will understand that too few binder locations 300 could result in inadequate bonding, or too much ‘loose’ fabric when the stent 101 is compressed, or that too many binder locations 305 could result in not achieving other desired benefits of selective bonding such as, without limitation, reducing radial forces when compressing a stent 101 or reducing resistance force to longitudinal extension or contraction during compressing or expansion, respectively, of a stent 101.

Even with some variations in the value of transverse location-location spacing LL_{TRANS}, including minor variations, it can be useful to set a minimum value for transverse location-location spacing LL_{TRANS}. This minimum value of LL_{TRANS} can apply to any transverse sections 300 of a given stent 101, or can vary with respect to different portions of a given stent 101, as might be the case for a stent 101 with variable diameter(s).In some embodiments, no LL_{TRANS} of a given transverse section 300 is greater than one-third of the circumference of the transverse section 300, or greater than one-quarter of the circumference of the transverse section 300, or greater than one-fifth of the circumference of the transverse section 300, or greater than one-sixth of the circumference of the transverse section 300, or greater than one-seventh of the circumference of the transverse section 300, or greater than one-eighth of the circumference of the transverse section 300. The foregoing minimum LL_{TRANS} values can be combined with the allowed LL_{TRANS} variation-ranges discussed hereinabove, such that the statement “no LL_{TRANS} of a given transverse section 300 is greater than one-third (for example) of the circumference of the transverse section 300” can be interpreted as “no LL_{TRANS} of a given transverse section 300 is greater than one-third of the circumference of the transverse section 300 ±5%, or ±10%, or ±15%, or ±20%, or ±25%, or ±30%”.

As shown in FIG. 18, fabric 106 can be bonded to the stent 101 by the selectively applied binder such that all, or at least 99%, or at least 95%, or at least 90%, or at least 85%, or at leas 80%, or at least 75%, of at least the circumference of at least the transverse section 300 is covered by the fabric 106. The stent assembly 100 of FIG. 18 shows the fabric bonded on the external (outwards-facing) major surface of the stent 101, but this is merely for illustration and in other examples the fabric can be bonded to the internal (inwards-facing) major surface of the stent 101.

A binder location 305 can merely include a spot, e.g., of a binder 104, or can include application of a binder 104 in any shape and any size. In the example of FIGS. 17A-17B, each location 305 includes a ‘splotch’ of binder 104 having no regular shape. In some examples, a location 305 can include a polygonal, e.g., rectangular, application of a binder 104 having an area of several square millimeters. In other examples, locations 305 can include specific shapes that are not polygonal.

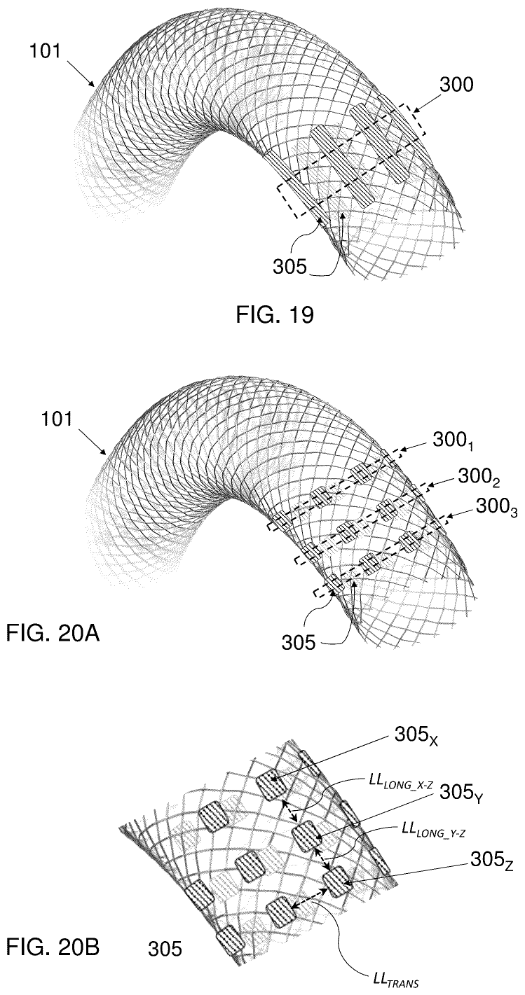

FIG. 19 shows another example of a stent 101 having a polymer applied at a number of locations 305 around a circumference of a transverse section 300, for selective bonding of a fabric to the stent 101. As can be seen in FIG. 19, the binder locations 305 can be elongated to any practical length which does overly not restrict the desired flexibility of the stent.

It can be desirable, in embodiments, to employ selective bonding in the longitudinal direction as well as the transverse direction, for example, to reduce the resistance to longitudinal extension/contraction of a stent when compressed/expanded (respectively). In such embodiments, rather than used elongated binder locations such as those shown in FIG. 19, it can be useful to apply the polymer binder 104 in multiple unconnected transverse-section ‘rings’ 300, for example, so as to further reduce the force required to compress the final stent assembly 100 (since the force applied typically must overcome longitudinal resistance as well as direct radial forces). Referring now to FIGS. 20A and 20B, multiple ring-like transverse sections 300₁, 300₂, 300₃ of a stent 101 are defined so that each encompasses a circumference of the stent 101 along at least a portion of a (longitudinal) length of a stent 101. Each transverse section 300 at least partially, a respective plurality of selectively-bonded binder locations 305 disposed circumferentially therearound. Three transverse sections 300₁, 300₂, 300₃ are shown for purposes of illustration but there can be any practical number of transverse sections 300 along the length of the stent 101. The transverse sections 300₁, 300₂, 300₃ can be contiguous or spaced-apart (as shown) but do not overlap. It should be understood that any or all of the features described in connection with the single transverse section 300 of FIGS. 17A and 17B (e.g., and not exhaustively: size, shape, spacing, etc.), can apply to each of the transverse sections 300₁, 300₂, 300₃.

As shown in the cutaway detail of FIG. 20B, binder locations 305 can be characterized not only by location-location spacing around the transverse circumference, i.e., LL_{TRANS}, but also by longitudinal location-location spacing LL_{LONG} along at least a portion of the length of the stent 101. Thus, the spacing between consecutive locations 305_{X} and 305_{Y} is LL_{LONG_X-Y} and the spacing between consecutive locations 305_{Y} and 305_{Z} is LL_{LONG_Y-Z}.

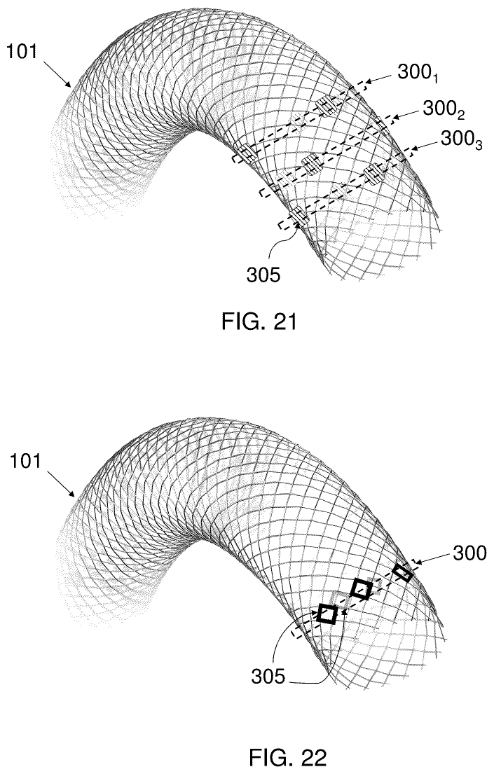

FIG. 21 shows a second example (after FIG. 20) of multiple transverse sections 300₁, 300₂, 300₃ of a stent 101. In this example, the binder locations 305 are ‘staggered’. In the non-limiting example of FIG. 20, three binder locations 305 are shown in each transverse section 300, although of course there can be more than three. The selective-binding ‘pattern’ repeats every other transverse section, such that the ‘pattern’ (positions of binder locations 305 around the circumference of the respective transverse section 300) of transverse section 300₁ is substantially the same as that of transverse section 300₃, and the circumferentially-offset patter of transverse section 300₂ is substantially the same as the ‘next’ transverse section (e.g., 300₄), which isn't shown because only 3 transverse sections 300 are shown in FIG. 21. Repeating selective-bonding binder-location ‘patterns’ can alternatively repeat, for example, with every third transverse section, or with every fourth transverse section, etc., with the underlying principle remaining that the requirement to provide reliable bonding of the fabric to the stent is combined with a desire to reduce, to a practical extent, the force required to compress the stent graft (and expand it, if the stent is not a self-expanding stent).

FIG. 22 illustrates yet another example of selective bonding, in which the polymer at each binder location 305 is applied so as to minimize the binder material in the ‘open’ areas of the stent 101 between the struts 102. While the embodiments disclosed herein can be applied with either porous or non-porous fabrics 106, in the case of porous fabrics it can desirable to reduce or minimize the extent to which fabric pores are clogged by the binder 104. Thus, it can be desirable to apply the polymer 104 in the binder locations 305 (as in the example of FIG. 22) such that not more than 30%, by area, of the fabric material disposed at each binder-location 305 and more than 0.5 mm from a nearest respective strut 102, is rendered non-porous by a presence of the polymer binder 104 within pores of the fabric material 102.

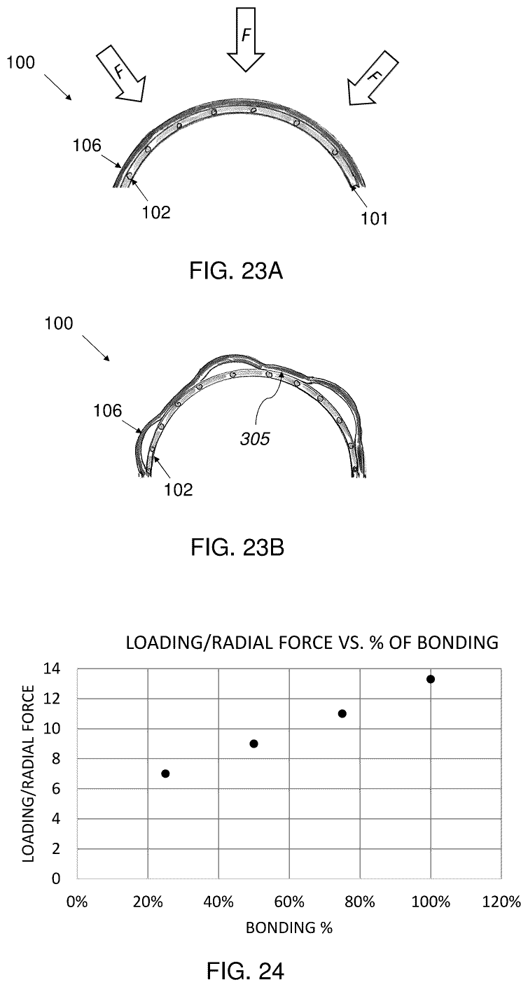

FIGS. 23A and 23B schematically illustrate radial forces (also known as ‘loading force’) applied to a stent assembly 100 for compression, e.g., for insertion into a delivery catheter. The forces shown in FIG. 23A are effective to compress the stent 101, and, as shown in FIG. 23B, the selective bonded of the fabric 106 (i.e., bonded at binder locations 305) reduces or minimizes wrinkling or bunching of the fabric 106 between the binder locations 305. To be clear, the terms ‘loading force’ and ‘radial forces’ are used herein interchangeably and for the purpose of this disclosure refer to the forces required for compressing a stent and/or the forces acting upon the lumen of a subject by an expanding (self-expanding or balloon-expanded) stent. The skilled artisan will understand that that the schematic illustration of radial forces in FIG. 23A is a simplification made for ease of presentation, and that numerous forces are at play in the sheathing, loading or compression of a stent, as well as in the in-site deployment and expansion. The forces can include, and not exhaustively, frictional and mechanical resistance forces during sheathing, loading and deploying; the resistance forces can include longitudinal resistance to extension and foreshortening or any change in length.

### Experimental Results

The extent to which selective bonding is effective to reduce the radial forces (as a representation of total forces as discussed in the preceding paragraph) required to compress a stent graft was tested for a number of selectively-bonded stent assemblies. The results of one illustrative experiment are shown in the graph of FIG. 24, which shows loading force, i.e., applied radial force (on a relative scale), as a function of bonding percentage, i.e., the percentage of the circumference of each transverse section 300 occupied in aggregate by binder locations 305.

In a first set of measurements, effective loading force was reduced by at least 20% when the occupied fraction of the circumference was at least 10% and not more than 50%. In a second set of measurements, the effective loading force was reduced by at least 25% when the fraction of the circumference was at least 10% and not more than 50%. In a third set of measurements, the effective loading force was reduced by at least 30% when the fraction of the circumference was at least 5% and not more than 25%. In a fourth set of measurements, the effective loading force was reduced by at least 35% when the fraction of the circumference was at least 5% and not more 25%. In a fifth set of measurements, the effective loading force was reduced by at least 40% when the fraction of the circumference was at least 5% and not more 25%. For all of the experimental measurements, the multiple binder-locations occupied, in aggregate, at least 5% and not more than 75% of the respective circumference of each transverse section.

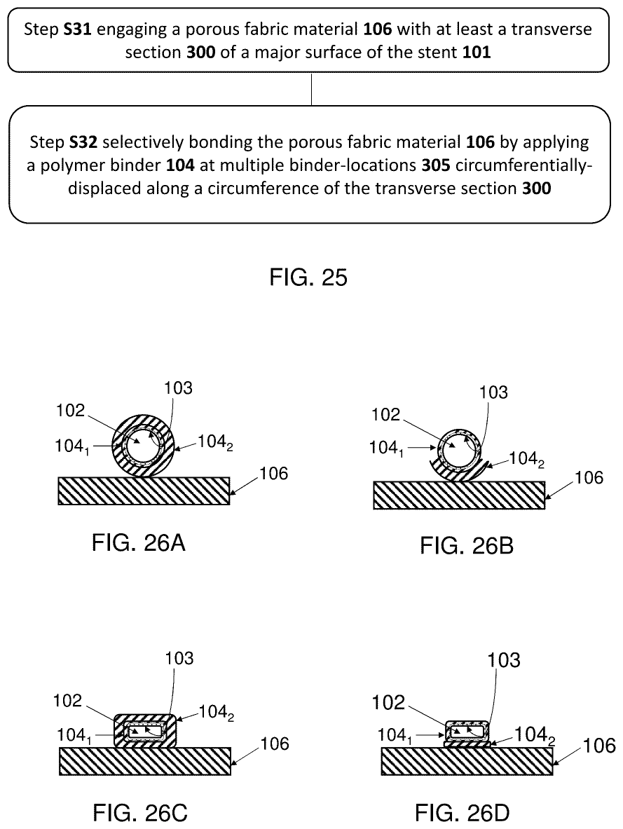

According to embodiments, a method is disclosed for attaching a fabric material to a stent formed by a network of struts. As illustrated in the flowchart of FIG. 25, the method comprises the following steps:

Step S31 engaging a porous fabric material 106 with at least a transverse section of a major surface (151 or 152) of the stent 101;

Step S32 selectively bonding the porous fabric material 106 to at least some struts 102 by applying a polymer binder 104 at multiple binder-locations 305 circumferentially-displaced along a circumference of the transverse section 300, wherein the multiple binder-locations 305 occupy, in aggregate, at least 5% and not more than 75% of the circumference of the transverse section 300, and the multiple binder-locations 300 are spaced such that no location-location spacing LL_{TRANS} is greater than one-third of the circumference of the transverse section 300.

We now refer to FIGS. 26A, 26B, 26C and 26D.

In embodiments, a strut 102, e.g., of a network of struts 102 making up a stent 101 or a portion of a stent 101, is surface-treated with a primer 103. The surface treatment with the primer 103 can include creating a covalent bond with the metallic or metal-alloy strut 102. A suitable, non-limiting example of surface treatment with a primer 103 is use of a primer such as cobalt acetoacetonate or triphenyl phosphine to increase polymerization rate of a cyanoacrylate adhesive. Another example of surface treatment with a primer 103 is silanization, where the primer 103 is a coupling agent that includes a reactive silane compound such as an organofunctional alkoxysilane compound. Following application of the primer/coupling agent 103, the strut 102 is encapsulated with a polymer binder 104 according to any of the examples of suitable polymer binders discussed hereinabove. The encapsulation is to a first binder thickness which can be between 1 micron and 10 microns, between 5 microns and 10 microns, between 1 micron and 5 microns, or within any range between a minimum thickness of at least 1 micron and a maximum thickness of no more than 10 microns (all ranges being inclusive). A second application of polymer binder 104, to a second binder thickness, can be used to selectively coat the strut 102 at multiple binder-locations 355 around the circumference of at least a transverse section 300 of the stent 101. Binder locations 355 are illustrated in FIGS. 17A, 17B, 18, 19, 20A, 20B, 21, and 22, and discussed hereinabove.

The embodiments illustrated in FIGS. 26A, 26B, 26C and 26D are specific examples of implementation of the embodiments and examples of selectively applying a binder at binder locations 355 as illustrated in FIGS. 17A, 17B, 18, 19, 20A, 20B, 21, and 22. The percentages of coverage of a circumference of a transverse section, the location-location spacing In these implementation examples, surfaces of struts 102, at least within respective transverse sections 300, are surface-treated by application of a binder 103, and encapsulated by application of the binder 104 to a first thickness. At the multiple binder-locations 355, the struts, already treated with the primer and encapsulated by the binder to a first thickness, are further selectively coated by application of the binder 104 to a second thickness. Thus, in such implementation examples, a strut 102 can be coated to the first binder thickness at least within one or more transverse sections 300, and selectively coated to the second binder thickness only at the binder-locations 355. The second binder application, i.e., the application of the binder to the second thickness, can include encapsulation or, alternatively, coating of only a portion of the circumference.

FIGS. 26A and 26B show cross-sections of a strut 102 having a circular cross-section, e.g., at a binder-location 355. A primer 103 has been applied to the strut 102 around its circumference. Examples of surface treatment with a primer 103 is use of a primer such as cobalt acetoacetonate or triphenyl phosphine to increase polymerization rate of a cyanoacrylate adhesive. Another example of a suitable primer is a primer/coupling agent comprising a silane compound so as to create a covalent bond with the strut 102 by silanization. The strut 102—with primer 103 is coated with a first application of the polymer binder 104₁ and is encapsulated by the binder 104₁ to a first thickness in any of the ranges disclosed hereinabove. A second application of the polymer binder 104₂ is applied to a second thickness that is at least twice the thickness of the first binder application 104₁ and which is in one of the ranges disclosed hereinabove. In the example of FIG. 26A, the second application of the polymer binder 104₂ is applied to encapsulate the stent 102 at the binder-location 355. In the example of FIG. 26B, the second application of the polymer binder 104₂ is applied to a portion of the circumference of the strut 102 to be bonded to the covering material 106.

FIGS. 26C and 26D show cross-sections of a strut 102 having a prismatic cross-section, e.g., at a binder-location 355. A primer 103 has been applied to the strut 102 around its circumference. The strut 102—with primer 103 is coated with a first application of the polymer binder 104₁ and is encapsulated by the binder 104₁ to a first thickness in any of the ranges disclosed hereinabove. A second application of the polymer binder 104₂ is applied to a second thickness that is at least twice the thickness of the first binder application 104₁ and which is in one of the ranges disclosed hereinabove. In the example of FIG. 26C, the second application of the polymer binder 104₂ is applied to encapsulate the stent 102 at the binder-location 355. In the example of FIG. 26B, the second application of the polymer binder 104₂ is applied to a portion of the periphery of the strut 102 to be bonded to the covering material 106.

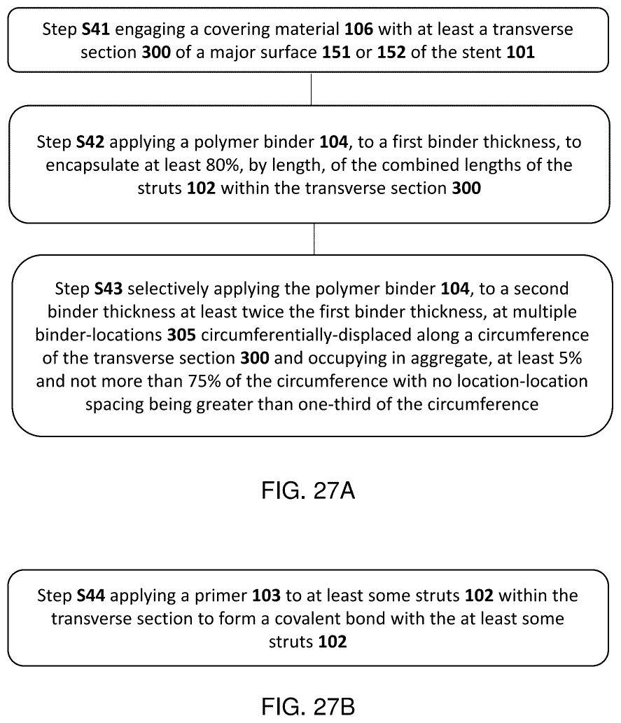

A method is disclosed, according to embodiments, for attaching a covering material 106 to a stent 101 formed by a network of struts 102. As seen in the flowchart of FIG. 27A, the method comprises:

Step S41 engaging a covering material 106, e.g., a fabric material, and/or an impermeable layer, and/or an impermeable elastomer and/or a non-woven material, with at least a transverse section 300 of a major surface 151 or 152 of the stent 101;

Step S42 applying a polymer binder 104₁, to a first binder thickness, to encapsulate at least 80%, by length, of the combined lengths of the struts 102 within the transverse section 300. In embodiments, the first binder thickness is not greater than 10 micron.

Step S43 selectively applying the polymer binder 104₂, to a second binder thickness at least twice the first binder thickness, at multiple binder-locations 305 circumferentially-displaced along a circumference of the transverse section 300 and occupying in aggregate, at least 5% and not more than 75% of the circumference, or not more than 50% of the circumference, or not more than 30 of the circumference, with no transverse location-location spacing LL_{TRANS} being greater than one-third of the circumference, or greater than one-quarter of the circumference, or greater than one-fifth of the circumference, or greater than one-sixth of the circumference. In embodiment, the second binder thickness is not greater than 40 microns.

In some embodiments, the method additionally comprises, as shown in the flowchart of FIG. 27B:

Step S44 applying a primer 103 to at least some struts 102 within the transverse section to form a covalent bond with the at least some struts 102.

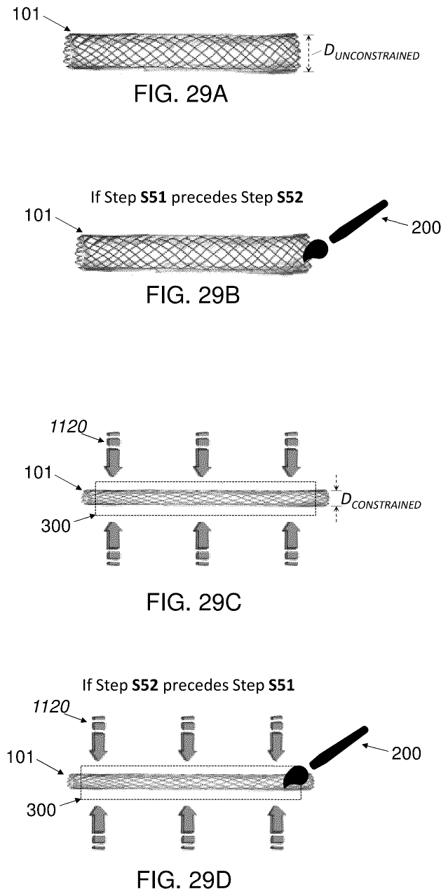

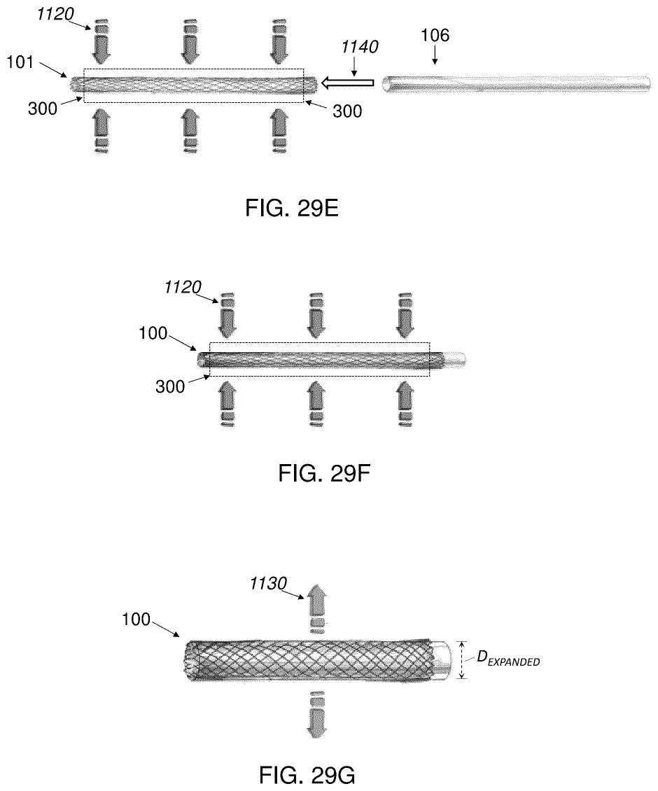

In some embodiments, Steps S43 and S44 are carried out before Step S41, and Step S41 includes radially constraining the transverse section 300 (as illustrated in FIG. 29A) so as to reduce a diameter thereof by at least 50%, and (ii) engaging the fabric while the transverse section is radially constrained (as illustrated in FIG. 29E). The fabric material 106 bonded to the at least some struts 102 can have an unfolded length along a circumference of the transverse section 300 that is no more than 20% greater than a circumference of the transverse section to 300 which the fabric material 106 is engaged.

In some embodiments Step S41 includes engaging a first covering material 106 with at least a transverse section 300 of a first major surface 151 or 152, and engaging a second covering material 106, different from the first covering material 106, with at least a transverse section 300 of a second major surface 152 or 151.

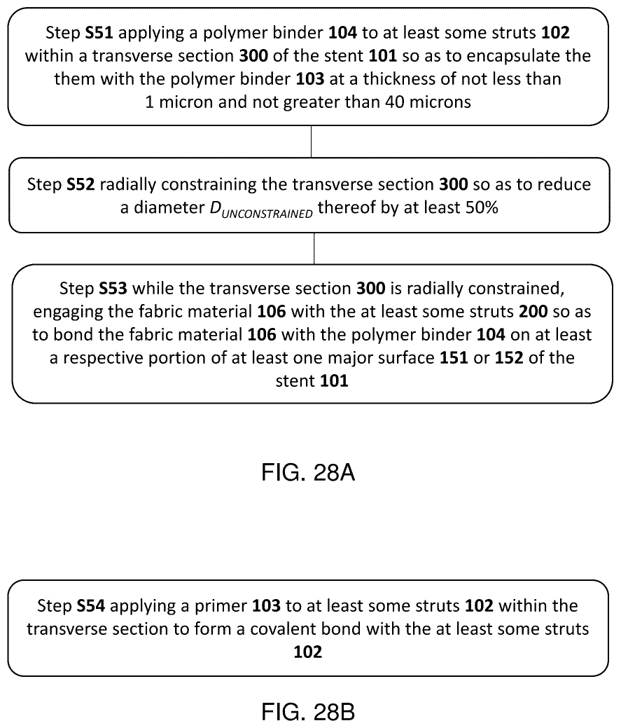

A method is disclosed, according to embodiments, attaching a fabric material 106 to a stent 101 comprising a metal alloy, e.g., a stainless steel or a nitinol, the stent 101 being formed by a network of struts 102 and having two major surfaces 151, 152. Performance of some of the method steps, and the results of performing some method steps, are shown schematically in FIGS. 29A, 29B, 29C, 29D, 29E, 29F and 29G. As seen in the flowchart of FIG. 28A, the method comprises:

Step S51 applying a polymer binder 104 to at least some struts 102 within a transverse section 300 of the stent 101 so as to encapsulate the them with the polymer binder 104 at a thickness of not less than 1 micron and not greater than 40 microns.

Step S52 radially constraining the transverse section 300 so as to reduce a diameter D_{UNCONSTRAINED} thereof by at least 50%.

Steps S51 and S52 can be performed in either order. As illustrated in FIG. 29A, a stent 101 has a diameter D_{UNCONSTRAINED} in an unconstrained state.

### If Step S51 Precedes Step S52

FIG. 29B shows, as per Step S51, application of the binder to the stent 101 as indicated schematically by paintbrush 200. In some embodiments, the application includes:
encapsulating the struts 102 of the stent 101, at least in transverse section 300, to a first binder thickness of no more than 10 micron, over at least 80%, by length, of the combined lengths of the struts 102 within the transverse section 300, and
selectively applying the polymer binder (not shown), to a second binder thickness that is at least twice the first binder thickness and no more than 40 microns, to at least some struts 102 within the transverse section 300. In some embodiments, the selectively applying the polymer binder is at multiple binder-locations circumferentially-displaced along a circumference of the transverse section, wherein the multiple binder-locations occupy, in aggregate, at least 5% and not more than 60% of the circumference, and the multiple binder-locations are spaced such that no location-location spacing is greater than one-third of the circumference.

Subsequently, in Step S52, the stent 101 is constrained, as shown schematically by constraining force 1120 in FIG. 29C, to have a diameter in a constrained state, or a reduced-diameter state, of D_{CONSTRAINED}, which is at least 50% less than unconstrained diameter D_{UNCONSTRAINED} shown in FIG. 29A. The constraining can be done in any tool or mechanical set-up suitable for providing a radial constraining force around the circumference and maintaining it as needed.

### If Step S52 Precedes Step S51

The stent 101 is constrained, as shown schematically by constraining force 1120 in FIG. 29C, to have a diameter in a constrained state, or a reduced-diameter state, of D_{CONSTRAINED}, which is at least 50% less than unconstrained diameter D_{UNCONSTRAINED} shown in FIG. 29A. The constraining can be done in any tool or mechanical set-up suitable for providing a radial constraining force around the circumference and maintaining it as needed.

Subsequently, as per Step S51 and as shown in FIG. 29D, application of the binder to the stent 101 as indicated schematically by paintbrush 200 while the stent 101 is constrained. In some embodiments, the application includes:
encapsulating the struts 102 of the stent 101, at least in transverse section 300, to a first binder thickness of no more than 10 micron, over at least 80%, by length, of the combined lengths of the struts 102 within the transverse section 300, and
selectively applying the polymer binder (not shown), to a second binder thickness that is at least twice the first binder thickness and no more than 40 microns, to at least some struts 102 within the transverse section 300. In some embodiments, the selectively applying the polymer binder is at multiple binder-locations circumferentially-displaced along a circumference of the transverse section, wherein the multiple binder-locations occupy, in aggregate, at least 5% and not more than 60% of the circumference, and the multiple binder-locations are spaced such that no location-location spacing is greater than one-third of the circumference.

Step S53 while the transverse section 300 is radially constrained, and as shown schematically in FIGS. 29E and 29F, engaging the fabric material 106 with the at least some struts 200 so as to bond the fabric material 106 with the polymer binder 104 on at least a respective portion of at least one major surface 151 or 152 of the stent 101 to form a stent assembly 100. In embodiments, the fabric material 106 engaged to the at least some struts 102 has an unfolded length along a circumference of the transverse section 300 that is no more than 20% greater than a circumference of the transverse section 300 to which the fabric material is engaged. In other words, no more than 20% excess fabric material is taken up in folds in the reduced-diameter state of the stent assembly 100.

In some embodiments, as shown in FIG. 28B, the method additionally comprises, before Step S51:

Step S54 applying a primer 103 to at least some struts 102 within the transverse section to form a covalent bond with the at least some struts 102. Application of the primer 103 between the at least some struts 102 within the transverse section 300 and the polymer binder can be effective to form a covalent bond with the at least some struts 102.

We now refer to FIG. 29G. In embodiments, the metal alloy of the struts 102 includes a shape-memory allow, and after cessation of the constraining of Steps S52 and S53, the force (indicated by arrow 1130) of self-expansion causes the diameter of the transverse section 300 to increase by at least 100% to an expanded diameter D_{EXPANDED}. Relative to the constrained diameter D_{CONSTRAINED} of the reduced-diameter state. In some embodiments, the diameter of the transverse section 300 increases by at least 200%.

In any of the embodiments disclosed herein, applying the polymer binder 104 can include extruding the binder 104.

The features of the embodiments disclosed herein can be usefully combined in combinations not specifically disclosed, and such combinations fall within the scope of the present invention.

The word ‘selectively’ as used in this disclosure and in the claims appended hereto refers to selected binder-locations for application of the polymer binding in at least one or more transverse sections, including: (a) applying only the binder at the selected specific binder-locations, (b) applying the binder at the selected specific binder-locations over a primer applied to the struts, e.g., throughout the at least one or more transverse sections, for surface treatments of the struts, and (c) applying the binder to a second thickness over a first thickness applied, e.g., throughout the at least one or more transverse sections, and over a primer applied directly to the struts, for surface treatments of the struts.

According to embodiments, a stent assembly comprises: (a) a stent formed by a network of struts, the stent having an internal surface and an external surface; (b) a covering material covering at least a transverse section of at least one of the internal surface and the external surface; and (c) a polymer binder mediating between the struts and the covering material to bind therebetween, the polymer binder (i) encapsulating at least 80%, by length, of the combined lengths of the struts of the network within the transverse section to a first binder thickness, and (ii) selectively having a second binder thickness that is at least twice the first binder thickness at multiple binder-locations circumferentially-displaced along a circumference of the transverse section and occupying in aggregate, at least 5% and not more than 75% of the circumference with no location-location spacing being greater than one-third of the circumference.

In some embodiments, it can be that the first binder thickness is not greater than 10 micron. In some embodiments, it can be that the second binder thickness is not greater than 40 microns.

In some embodiments, at least a portion of the covering material can include an impermeable material layer. In some embodiments, the covering material can include an impermeable elastomer. In some embodiments, the covering material can include a non-woven material. In some embodiments, the covering material can include a woven fabric.

In some embodiments, the stent assembly can additionally include a primer between at least some struts of the network within the transverse section and the polymer binder to form a covalent bond with the at least some struts.

In some embodiments, it can be that the stent assembly has a fully-expanded state in the absence of a radial constraint and a reduced-diameter state characterized by the stent being radially constrained to reduce a diameter of the stent by at least 50%, wherein when the stent assembly is in the reduced-diameter state, the covering material covering the transverse section has an unfolded length along a circumference of the transverse section that is no more than 20% greater than a circumference of the covered transverse section.

In some embodiments, it can be that no location-location spacing is greater than one-quarter of the circumference. In some embodiments, it can be that no location-location spacing is greater than one-fifth of the circumference. In some embodiments, it can be that no location-location spacing is greater than one-sixth of the circumference. In some embodiments, the multiple binder-locations can occupy, in aggregate, not more than 50% of the circumference. In some embodiments, the multiple binder-locations can occupy, in aggregate, not more than 30% of the circumference.

In some embodiments, the stent assembly can include a first covering material covering at least a transverse section of the internal surface, and a second covering material, different from the first covering material, covering at least a transverse section of the external surface.

A method is disclosed, for attaching a covering material to a stent formed by a network of struts. The method comprises: (a) engaging a covering material with at least a transverse section of a major surface of the stent; (b) applying a polymer binder, to a first binder thickness, so as to encapsulate at least 80%, by length, of the combined lengths of the struts of the network within the transverse section; and (c) selectively applying the polymer binder, to a second binder thickness that is at least twice the first binder thickness, at multiple binder-locations circumferentially-displaced a long a circumference of the transverse section and occupying in aggregate, at least 5% and not more than 75% of the circumference with no location-location spacing being greater than one-third of the circumference.

In some embodiments, it can be that the first binder thickness is not greater than 10 micron. In some embodiments, it can be that the second binder thickness is not greater than 40 microns.

In some embodiments, the method of can additionally include, before the applying the polymer binder: applying a primer to at least some struts of the network within the transverse section to form a covalent bond with the at least some struts.

In some embodiments, the applying the polymer binder and the selectively applying the polymer binder can be carried out before the engaging the covering material, and/or the engaging the covering material can include: (i) radially constraining the transverse section so as to reduce a diameter thereof by at least 50%, and (ii) engaging the covering material while the transverse section is radially constrained.

In some embodiments, the covering material can be bonded to the at least some struts has an unfolded length along a circumference of the transverse section that is no more than 20% greater than a circumference of the transverse section to which the covering material is engaged.

In some embodiments, upon cessation of the radial constraining, a diameter of the transverse section can increase by at least 100%. In some embodiments, upon cessation of the radial constraining, a diameter of the transverse section can increase by at least 200%.

In some embodiments, it can be that no location-location spacing is greater than one-quarter of the circumference. In some embodiments, it can be that no location-location spacing is greater than one-fifth of the circumference. In some embodiments, it can be that no location-location spacing is greater than one-sixth of the circumference.

In some embodiments, it can be that the multiple binder-locations occupy, in aggregate, not more than 50% of the circumference. In some embodiments, it can be that the multiple binder-locations occupy, in aggregate, not more than 30% of the circumference.

In some embodiments, the engaging the covering material can include engaging a first covering material with at least a transverse section of a first major surface, and engaging a second covering material, different from the first covering material, with at least a transverse section of a second major surface.

In some embodiments, at least a portion of the covering material can include an impermeable material layer. In some embodiments, the covering material can include an impermeable elastomer. In some embodiments, the covering material can include a non-woven material. In some embodiments, the covering material can include a woven fabric.

According to embodiments, a stent assembly comprises (a) a stent formed by a network of struts, the stent having an internal surface and an external surface; and (b) a fabric material covering at least a transverse section of at least one of the internal surface and the external surface, and bonded to the stent by a polymer binder that mediates between the fabric material and at least some struts of the network of struts to encapsulate the at least some struts and bind between the at least some struts and the fabric material at multiple binder-locations circumferentially-displaced along a circumference of the transverse section, wherein a loading force effective to compress the transverse section from an expanded state to a compressed state is a function of the fraction of the circumference occupied, in aggregate, by the multiple binder-locations, such that the effective loading force is reduced by at least 20% when the occupied fraction of the circumference is at least 10% and not more than 50%.

In some embodiments, it can be that the effective loading force is reduced by at least 25% when the fraction of the circumference is at least 10% and not more than 50%. In some embodiments, it can be that the effective loading force is reduced by at least 30% when the fraction of the circumference is at least 5% and not more than 25%. In some embodiments, it can be that the effective loading force is reduced by at least 35% when the fraction of the circumference is at least 5% and not more 25%. In some embodiments, it can be that the effective loading force is reduced by at least 40% when the fraction of the circumference is at least 5% and not more 25%.

In some embodiments, it can be that the multiple binder-locations occupy, in aggregate, at least 5% and not more than 75% of the circumference. In some embodiments, it can be that the multiple binder-locations are spaced such that no location-location spacing is greater than one-third of the circumference.

In some embodiments, it can be that no location-location spacing is greater than one-quarter of the circumference. In some embodiments, it can be that no location-location spacing is greater than one-fifth of the circumference. In some embodiments, it can be that no location-location spacing is greater than one-sixth of the circumference.

In some embodiments, it can be that the multiple binder-locations occupy, in aggregate, not more than 50% of the circumference. In some embodiments, it can be that the multiple binder-locations occupy, in aggregate, not more than 30% of the circumference.

In some embodiments, all of the respective location-location spacings can be the same, or within ±30% of each other, or within ±25% of each other, or within ±20% of each other, or within ±15% of each other, or within ±10% of each other, or within ±5% of each other.

In some embodiments, the substantially complete circumference can include at least 95% of a circumference, or at least 90% of a circumference, or at least 85% of a circumference, or at least 80% of a circumference or at least 75% of a circumference.

In some embodiments, the stent assembly can include a first fabric material covering at least a transverse section of the internal surface, and a second fabric material, different from the first fabric material, covering at least a transverse section of the external surface.

A method is disclosed, according to embodiments, for attaching a fabric material to a stent comprising a metal alloy, the stent being formed by a network of struts and having two major surfaces. The method comprises: (a) applying a polymer binder to at least some struts within a transverse section of the stent so as to encapsulate the at least some struts with the polymer binder at a polymer-binder thickness of not less than 1 micron and not greater than 40 microns; (b) radially constraining the transverse section so as to reduce a diameter thereof by at least 50%; and (c) while the transverse section is radially constrained, engaging the fabric material with the at least some struts so as to bond the fabric material with the polymer binder on at least a respective portion of at least one major surface of the stent.

In some embodiments, the applying the polymer binder can include: (i) encapsulating, to a first binder thickness of no more than 10 micron, at least 80%, by length, of the combined lengths of the struts of the network within the transverse section, and (ii) selectively applying the polymer binder, to a second binder thickness that is at least twice the first binder thickness and no more than 40 microns, to at least some struts within the transverse section.

In some embodiments, the transverse section can be is in a fully-expanded state during the applying of the polymer binder.

In some embodiments, the method can additionally include, before the applying the polymer binder: applying a primer to at least some struts of the network within the transverse section to form a covalent bond with the at least some struts.

In some embodiments, it can be that the applying of the polymer binding includes extruding the polymer binder.

In some embodiments, it can be that the fabric material engaged to the at least some struts can have an unfolded length along a circumference of the transverse section that is no more than 20% greater than a circumference of the transverse section to which the fabric material is engaged.

In some embodiments, upon cessation of the radial constraining, the diameter of the transverse section can increase by at least 100%. In some embodiments, upon cessation of the radial constraining, the diameter of the transverse section can increase by at least 200%.

In some embodiments, it can be that (i) the selectively applying the polymer binder is at multiple binder-locations circumferentially-displaced along a circumference of the transverse section, (ii) the multiple binder-locations occupy, in aggregate, at least 5% and not more than 75% of the circumference, and/or (iii) the multiple binder-locations are spaced such that no location-location spacing is greater than one-third of the circumference.

In some embodiments, it can be that at the reduced circumference, a diameter of the stent is no more than 2 mm.

According to embodiments, a stent assembly comprises: (a) a stent formed by a network of struts, the stent having an internal surface and an external surface; and (b) a fabric material covering at least a transverse section of at least one of the internal surface and the external surface, and bonded to the stent by a polymer binder that mediates between the fabric material and at least some struts of the network of struts to bind therebetween. The stent assembly has a fully-expanded state in the absence of a radial constraint and a reduced-diameter state characterized by the stent being radially constrained to reduce a diameter of the stent by at least 50%; when the stent assembly is in the reduced-diameter state, the fabric material covering the transverse section has an unfolded length along a circumference of the transverse section that is no more than 20% greater than a circumference of the covered transverse section.

In some embodiments, it can be that the polymer binder (i) encapsulates at least 80%, by length, of the combined lengths of the struts of the network within the transverse section to a first binder thickness, and/or (ii) selectively has a second binder thickness that is at least twice the first binder thickness at multiple binder-locations circumferentially-displaced along a circumference of the transverse section and occupying in aggregate, at least 5% and not more than 75% of the circumference with no location-location spacing being greater than one-third of the circumference.

In some embodiments, it can be that the first binder thickness is not greater than 10 micron. In some embodiments, it can be that the second binder thickness is not greater than 40 microns.

In some embodiments, the stent assembly can additionally include a primer between at least some struts of the network within the transverse section and the polymer binder to form a covalent bond with the at least some struts. In some embodiments, the primer can comprise a coupling agent.

In embodiments, a stent assembly comprises a stent formed by a network of strut-segments. The stent generally manifests as a hollow tube or other three-dimensional structure, and thus includes an internal surface and an external surface. Each strut-segment has a transverse cross-section defining a respective shape. In some embodiments, sufficient adhesion of a fabric material to a stent scaffold is achieved by coating and covering a tangential section of a strut perimeter, for example as illustrated in FIGS. 7A and 12A. It is now disclosed that substantially more robust adhesion, especially suitable for stent grafts which undergo bending and/or expansion in situ, is achievable by applying a coating, and engaging therewith the fabric material covering, with a larger transverse portion of the perimeter of the strut, and preferably a much larger transverse portion than the ‘tangential’ application illustrated in FIGS. 7A and 12A. It is further disclosed that struts or strut-segments having prismatic cross-sectional shapes generally require a larger transverse portion to be coated and covered than struts or strut-segments having rounded shapes, e.g., to achieve a desired level of robustness of adhesion.

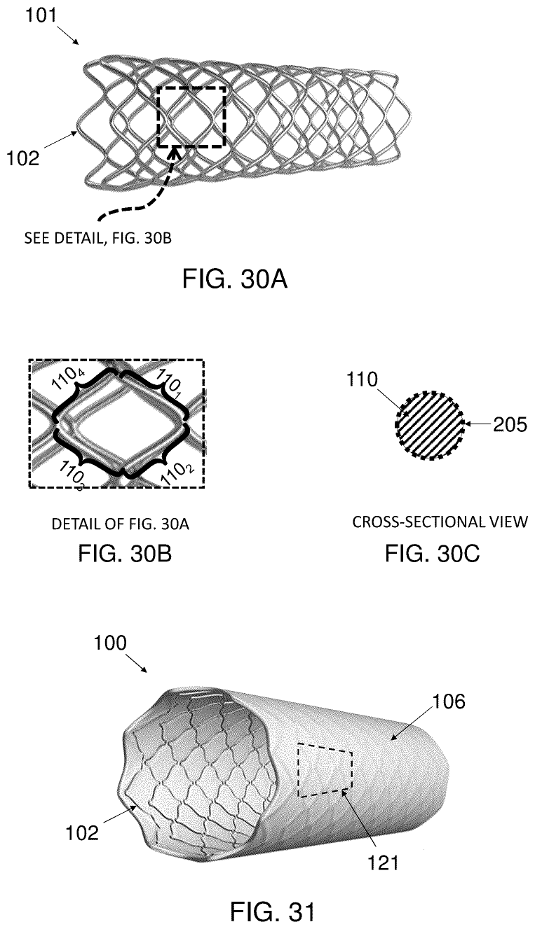

Referring again to the figures, and in particular to FIGS. 30A, 30B, and 30C, a stent scaffold 101 (stent without fabric cover) is formed by or from a network of strut-segments 110. An exemplary strut-segment 110 has a transverse cross-section having a rounded shape, i.e., the transverse cross-section has a perimeter 205 with a rounded shape. Stent-segment cross-sections can have varied shapes, which are generally categorizable as being rounded or prismatic. FIG. 31 shows a stent assembly 100 (covered stent, or stent graft) according to embodiments, and identifies a given area 121 that includes a region of the stent 101. A given area 121 can be selected for purposes of defining a set of features or characteristics of interest and can include any area comprising at least two strut-segments 110. In embodiments, a given area 121 can be characterized by all the strut-segments 110 therewithin having the same cross-sectional shapes. FIG. 31 shows a non-limiting example of shape, size and location of a given area 121 on a stent assembly 100, and in other examples, a given area can be larger or smaller, of any practical shape, and located anywhere on the stent assembly 100.

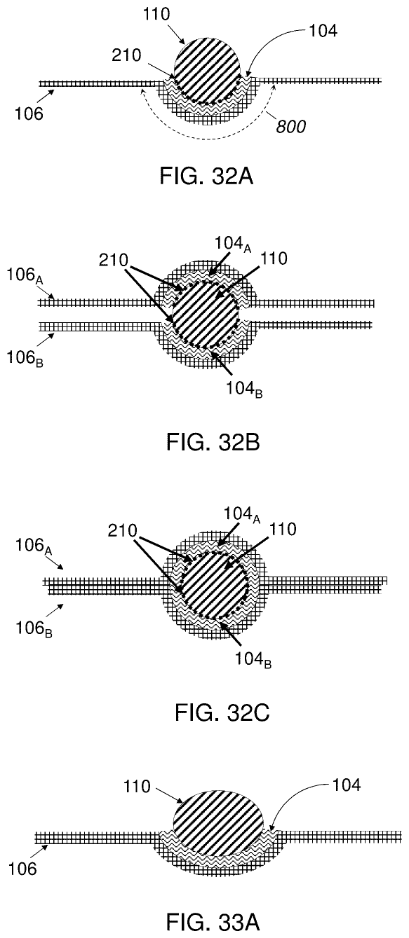

A stent assembly 100, as illustrated in FIG. 31, comprises the stent 101, a polymer binder 104 applied to strut-segments 110 of the stent 101 and a fabric material 106 covering (at least) a given part 121 of the stent 101 on at least a first surface selected from the internal surface and the external surface. Where the fabric material 106 covers a surface of a given area 121 of the stent 101 on either or both of the surfaces 152, 151 of the stent 101, the fabric material 106 is bonded to each strut-segment 110 within the given part 121 by a polymer binder 104. Specifically, the fabric material 106 is bonded by the polymer binder 104 to a respective transverse portion 210 of the perimeter 205 of each of the strut-segments 110 in the given area 121, as illustrated in FIG. 32A.

FIG. 32A is a schematic cross-sectional view of a strut-segment 110 having a circular shape, i.e., a rounded shape, as in FIG. 30C. The polymer binder coating 104 and fabric material 106 are applied to and engaged with, respectively, to a transverse portion 210 of the perimeter 205 of the stent-segment 110 on a single surface of the stent (either the external surface 152 or the internal surface 151), as indicated by arrow 800. In embodiments, the arc traversed by the transverse portions 210 is at least 30%, or at least 35%, or at least 40%, or at least 45%, or at least 50%, of the perimeter 205—of strut-segments 110 having a transverse cross-section defining a rounded shape. In some embodiments, the arc traversed by the transverse portions 210 is at least 40% of the perimeter 205. In embodiments, all of the strut-segments 110 in a given area 121 have the same transverse cross-section.

FIG. 32B illustrates the same or similar circular/rounded strut-segment 110, with both surfaces 152, 151 of the stent 101 in the given area 121 coated with a respective layer of the polymer binder 104A, 104B and covered on both surfaces 152, 151 by a respective fabric material 106A, 106B. In this non-limiting example, the transverse portion 210 is discontinuous, as it includes two different and non-contiguous transverse portions 210 of the perimeter 205. In some embodiments, all of the transverse portions 210 within the given area 121 are discontinuous. In other examples, not illustrated, the coating with the polymer binder can be done to one continuous transverse portion 210, as illustrated in FIGS. 32C. In embodiments, the sum of the one or more arcs traversed by the transverse portion 210 is at least 70%, or at least 75%, or at least 80%, or at least 85%, or at least 90%, of the perimeter 205—of strut-segments 110 having a transverse cross-section defining a rounded shape. In some embodiments, the total of the one or more arcs traversed by the transverse portions 210 are at least 80% of the perimeter 205.

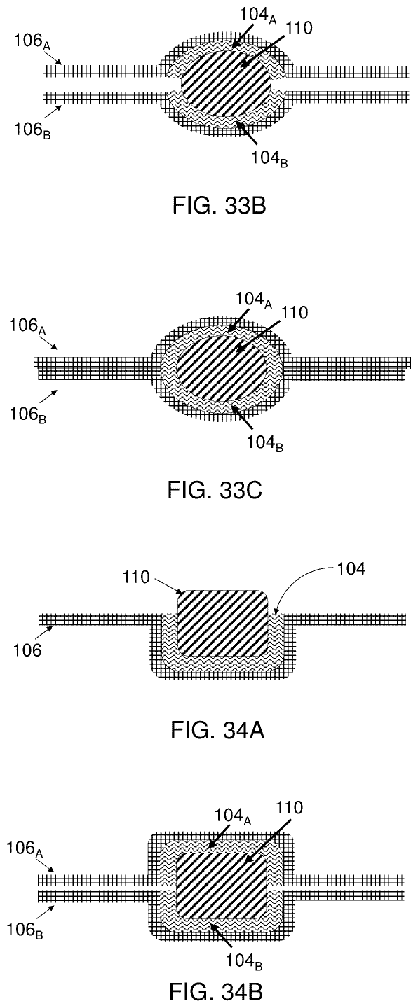

FIGS. 33A, 33B and 33C are similar to FIGS. 32A, 32B and 33C, respectively, but relate to strut-segments 110 having an oval or elliptical cross sectional shape rather than the circular shape of FIGS. 32A, 32B and 32C. The ratios of the transverse portion 210 relative to the perimeter 205 is the same as in the example of FIGS. 32A, 32B and 32C based on both shapes being rounded shapes.

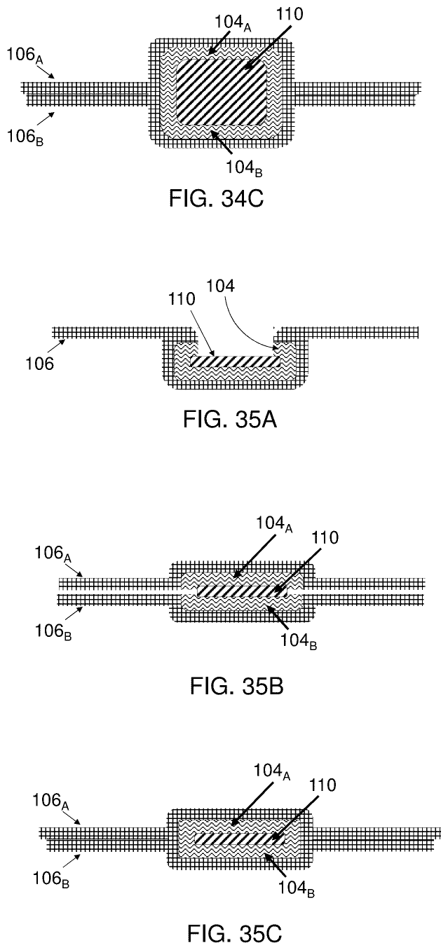

FIGS. 34A, 34B, 34C 35A, 35B and 35C relate to strut-segments 110 having prismatic cross-sectional shapes. In embodiments, the transverse portion 210 of prismatic-shaped strut-segments comprises a larger proportion of the perimeter than does the transverse portion 210 of rounded-shaped strut segments. Thus, the coverage proportion of the transverse portions 210 when the given area 121 includes strut-segments covered on a single surface of the stent (either the external surface 152 or the internal surface 151) as illustrated in FIGS. 34A and 35A is at least 55%, or at least 60%, or at least 65%, or at least 70%, of the perimeter 205—of the strut-segments 110 in the given area 121 having a transverse cross-section defining a prismatic shape. The aggregate coverage proportion of the transverse portions 210 when the given area 121 includes strut-segments covered on both the external and internal surfaces 152, 151, as illustrated in FIGS. 34B and 35B, is at least 80%, or at least 85%, or at least 90%, or at least 95%, or at least 70%, of the perimeter 205—of the strut-segments 110 having a transverse cross-section defining a prismatic shape.

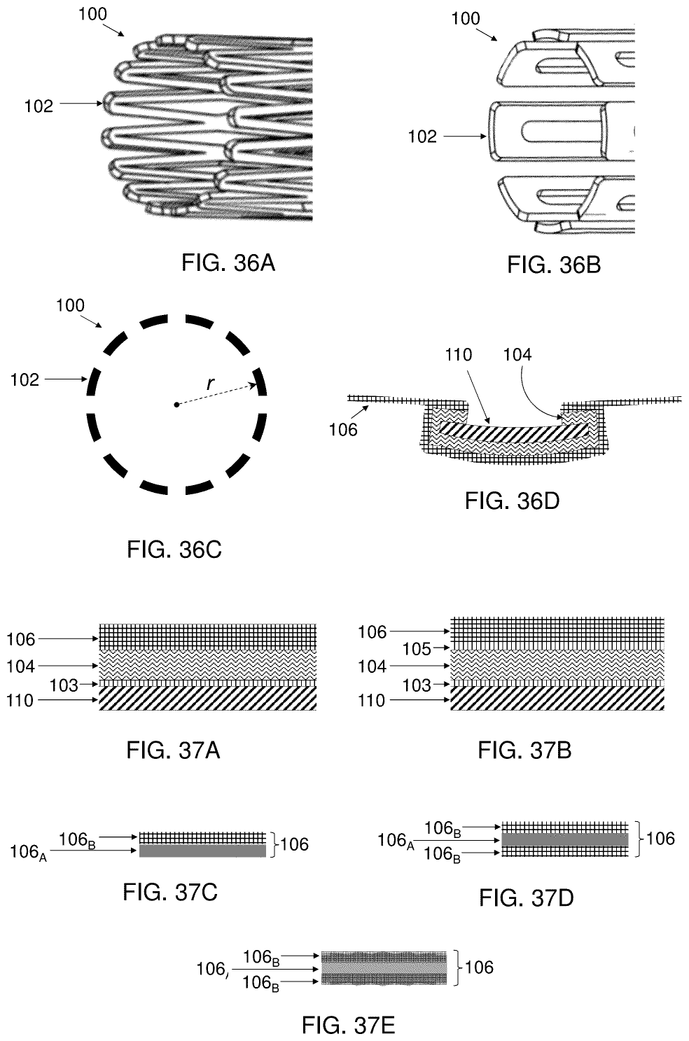

FIGS. 36A, 36B, 36C, and 36D are illustrative of differentiating between strut-segments having a transverse cross-section defining a rounded shape and strut-segments having a transverse cross-section defining a prismatic shape. FIGS. 36A and 36B are illustrative examples of stents whose struts have ‘curved prismatic shapes’, i.e., are curved, but not necessarily rounded. The distinction is explained in FIG. 36C: where a strut cross-section has a radius r that is equal to or greater than the radius of the stent 100, the strut 102 can be considered as prismatic for the purpose of determining a minimum proportion for a transverse portion 210 (relative to the perimeter 205), e.g., for desired robustness of adhesion of the fabric material 106. Conversely, if a strut cross-section has a radius r that is smaller than the radius of the stent 100, the strut 102 can be considered as rounded for the purpose of determining a minimum proportion for a transverse portion 210 (relative to the perimeter 205), e.g., the lower minimum ratios discussed above for rounded strut shapes that are sufficient to achieve the desired robustness of adhesion of the fabric material 106 to the strut-segments 110. FIG. 36D shows an illustrative, non-limiting example, in transverse cross-section, of such a strut-segment 110 and the requisite costing (by binder 104) and covering (by fabric 106) over the larger transverse portion 210 of a prismatic-shaped strut-segment.

FIGS. 37A and 37B show a more detailed view of the structure of a stent assembly 100. In the non-limiting example of FIG. 37A, a strut-segment 110 is treated with a coupling agent 103 before coating with polymer binder 104. The coupling agent 103 mediating between the polymer binder 104 and the respective transverse portion 210 is effective to create a covalent bond between the polymer binder 104 and the respective transverse portion 210. FIG. 37B shows another non-limiting example in which a second coupling agent 205 mediating between the polymer binder 104 and the fabric material 106 is effective to create a covalent bond between the polymer binder 104 and the fabric material 106. The second coupling agent 205 may or may not be the same coupling agent as coupling agent 203.

FIGS. 37C and 37D show further detail of the fabric material 106 according to examples in which the fabric material comprising multiple layers of material. FIG. 37C shows an example in which the fabric material 106 comprises a substantially impermeable layer 106A, comprising, for example, a non-woven fabric, and a permeable or porous layer 106B, comprising, for example, a woven or expanded fabric. The porous layer 106B can be disposed ‘above’ or ‘below’ the impermeable layer 106A, i.e., relative to the strut segment 110. FIG. 37D shows an example in which the fabric material 106 comprises a substantially impermeable layer 106A, comprising, for example, a non-woven fabric, and two permeable or porous layers 106B, comprising, for example, a woven or expanded fabric. The respective porous layers 106B are disposed ‘above’ and ‘below’ the impermeable layer 106A, i.e., relative to the strut segment 110. As shown in FIG. 37E, the material of the impermeable layer can, upon application of heat and/or pressure, flow into and through pores or other spaces in the one or more permeable layers 106B, and thereby improve bonding of the fabric layer 106 to the layers below, including the strut segment 110.

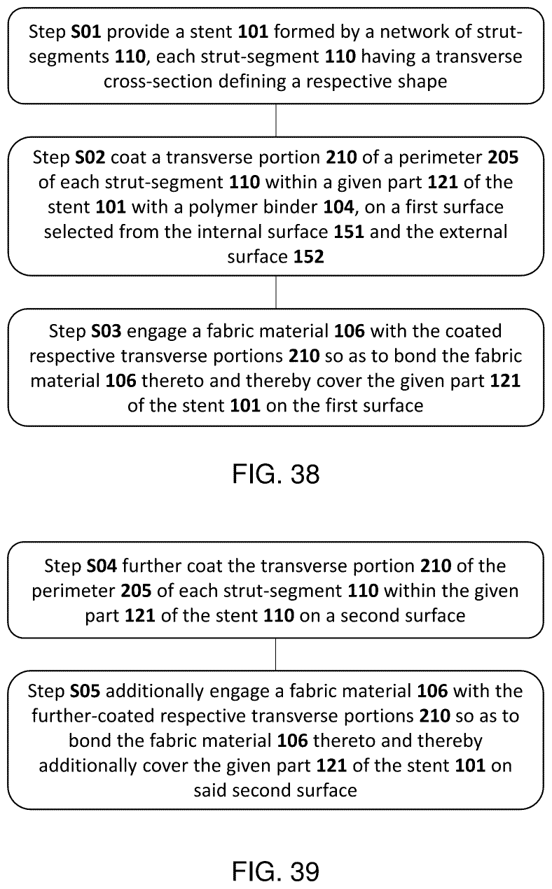

Referring now to FIG. 38, a method is disclosed assembling a stent assembly. As illustrated by the flowchart in FIG. 38, the method comprises at least the following steps S01, S02 and S03:

Step S01: providing a stent 101 formed by a network of strut-segments 110, each strut-segment 110 having a transverse cross-section defining a respective shape, e.g., prismatic or rounded. Any of the stents 101 disclosed herein are suitable for use in Step S01.

Step S02: coating a transverse portion 210 of a perimeter 205 of each strut-segment 110 within a given part 121 of the stent 101 with a polymer binder 104, on a first surface selected from the internal surface 151 and the external surface 152. The coated respective transverse portion 210 comprises at least 30%, or at least 35%, or at least 40%, or at least 45%, or at least 50%, of the respective perimeter 205 of each strut-segment 110 having a transverse cross-section defining a rounded shape, and at least 55%, or at least 60%, or at least 65%, or at least 70%, of the respective perimeter 210 of each strut-segment 110 having a transverse cross-section defining a prismatic shape. In some embodiments, each strut-segment 110 within the given area 121 has a transverse cross-section defining a rounded shape, and the respective transverse portion 210 comprises at least 40% of the respective perimeter 205 of each strut-segment 110. In some embodiments, each strut-segment 110 within the given area 121 has a transverse cross-section defining a prismatic shape, and the respective transverse portion 210 comprises at least 60% of the respective perimeter 205 of each strut-segment 110. In some embodiments, at least one of the transverse portions 210 in the given area 121 is discontinuous, and in some embodiments all of the transverse portions 210 in the given area 121 are discontinuous. In some embodiments, Step S02 includes applying a coupling agent 103 between the polymer binder 104 and the respective transverse portions 210 to form a covalent bond therebetween.

Step S03: engaging a fabric material 106 with the coated respective transverse portions 210 so as to bond the fabric material 106 thereto and thereby cover the given part 121 of the stent 101 on the first surface. In some embodiments, Step S03 includes applying a coupling agent 103 to mediate between the polymer binder 104 and the fabric material 106 to form a covalent bond therebetween.

In some embodiments, the method includes two additional steps S04 and S05, as illustrated in FIG. 39:

Step S04: further coating the transverse portion 210 of the perimeter 205 of each strut-segment 110 within the given part 121 of the stent 110 on a second surface.

The respective transverse portion 210 comprises at least 70%, or at least 75%, or at least 80%, or at least 85%, or at least 90%, of the respective perimeter 205 of each strut-segment 110 having a transverse cross-section defining a rounded shape, and at least 80%, or at least 85%, or at least 90%, or at least 95%, of the respective perimeter 205 of each strut-segment having a transverse cross-section defining a prismatic shape. In some embodiments, each strut-segment 110 within the given area 121 has a transverse cross-section defining a rounded shape, and the respective transverse portion 210 comprises at least 80% of the respective perimeter 205 of each strut-segment. In some embodiments, each strut-segment 110 within the given area 121 has a transverse cross-section defining a prismatic shape, and the respective transverse portion 210 comprises at least 90% of the respective perimeter 205 of each strut-segment. In some embodiments, Step S04 includes applying a coupling agent 103 between the polymer binder 104 and the respective transverse portions 210 to form a covalent bond therebetween. In some embodiments, Step S05 includes applying a coupling agent 103 to mediate between the polymer binder 104 and the fabric material 106, e.g., the fabric material covering the second stent surface, to form a covalent bond therebetween.

Step S05: additionally engaging a fabric material 106 with the further-coated respective transverse portions 210 so as to bond the fabric material 106 thereto and thereby additionally cover the given part 121 of the stent 101 on said second surface

Any of the method steps can be combined with any other method steps within the scope of the present invention. Not all method steps need be carried out in order to practice the methods.

### ADDITIONAL DISCUSSION

The following paragraphs summarize inventive concepts discussed in this disclosure, numbered for convenience.

Inventive concept 1. A stent assembly comprising:
a. a stent formed by a network of strut-segments and comprising an internal surface and an external surface, each strut-segment having a transverse cross-section defining a respective shape;
b. a fabric material covering a given part of the stent on a first surface selected from the internal surface and the external surface; and
c. a polymer binder,
wherein
i. the covering by the fabric material is such that the fabric material is bonded to a respective transverse portion of a perimeter of each strut-segment within the given part by the polymer binder, and
ii. the respective transverse portion comprises at least 30% of the respective perimeter of each strut-segment having a transverse cross-section defining a rounded shape, and at least 55% of the respective perimeter of each strut-segment having a transverse cross-section defining a prismatic shape.

Inventive concept 2. The stent assembly of Inventive concept 1, wherein (i) the fabric material additionally covers the given part on a second surface, such that both the internal surface and the external surface of the given part are covered, and (ii) the respective transverse portion comprises at least 70% of the respective perimeter of each strut-segment having a transverse cross-section defining a rounded shape, and at least 80% of the respective perimeter of each strut-segment having a transverse cross-section defining a prismatic shape.

Inventive concept 3. The stent assembly of Inventive concept 1, wherein each strut-segment within the given area has a transverse cross-section defining a rounded shape, and the respective transverse portion comprises at least 40% of the respective perimeter of each strut-segment.

Inventive concept 4. The stent assembly of Inventive concept 1, wherein each strut-segment within the given area has a transverse cross-section defining a prismatic shape, and the respective transverse portion comprises at least 60% of the respective perimeter of each strut-segment.

Inventive concept 5. The stent assembly of Inventive concept 2, wherein each strut-segment within the given area has a transverse cross-section defining a rounded shape, and the respective transverse portion comprises at least 80% of the respective perimeter of each strut-segment.

Inventive concept 6. The stent assembly of Inventive concept 2, wherein each strut-segment within the given area has a transverse cross-section defining a prismatic shape, and the respective transverse portion comprises at least 90% of the respective perimeter of each strut-segment.

Inventive concept 7. The stent assembly of any one of Inventive concepts 2 to 6, wherein the fabric material covering the given part of the stent on the first surface and the fabric material covering the given part of the stent on the second surface are in contact with each other.

Inventive concept 8. The stent assembly of any one of the preceding Inventive concepts, wherein at least one respective transverse portion is discontinuous.

Inventive concept 9. The stent assembly of any one of Inventive concepts 2 to 8, wherein all of the respective transverse portions are discontinuous.

Inventive concept 10. The stent assembly of any one of the preceding Inventive concepts, additionally comprising a coupling agent mediating between the polymer binder and the respective transverse portions.

Inventive concept 11. The stent assembly of Inventive concept 10, wherein the coupling agent is effective to create a covalent bond between the polymer binder and the respective transverse portions.

Inventive concept 12. The stent assembly of any one of the preceding Inventive concepts, additionally comprising a coupling agent mediating between the polymer binder and the fabric material.

Inventive concept 13. The stent assembly of Inventive concept 12, wherein the coupling agent is effective to create a covalent bond between the polymer binder and the fabric material.

Inventive concept 14. The stent assembly of any one of the preceding Inventive concepts, wherein the fabric material comprises a substantially impermeable layer and at least one permeable layer.

Inventive concept 15. The stent assembly of Inventive concept 14, wherein a material of the substantially impermeable layer is made to enter spaces of the at least one permeable layer.

Inventive concept 16. A method for assembling a stent assembly, the method comprising:
a. providing a stent that comprises an internal surface and an external surface, and that is formed by a network of strut-segments, each strut-segment having a transverse cross-section defining a respective shape;
b. coating, with a polymer binder, a respective transverse portion of a perimeter of each strut-segment within a given part of the stent on a first surface selected from the internal surface and the external surface, wherein the coated respective transverse portion comprises at least 30% of the respective perimeter of each strut-segment having a transverse cross-section defining a rounded shape, and at least 55% of the respective perimeter of each strut-segment having a transverse cross-section defining a prismatic shape; and
c. engaging a fabric material with the coated respective transverse portions so as to bond the fabric material thereto and thereby cover the given part of the stent on the first surface.

Inventive concept 17. The method of Inventive concept 16, additionally comprising:
i. further coating, with the polymer binder, the respective transverse portion of the perimeter of each strut-segment within the given part of the stent on a second surface, wherein the respective transverse portion comprises at least 70% of the respective perimeter of each strut-segment having a transverse cross-section defining a rounded shape, and at least 80% of the respective perimeter of each strut-segment having a transverse cross-section defining a prismatic shape, and
ii. additionally engaging a fabric material with the further-coated respective transverse portions so as to bond the fabric material thereto and thereby additionally cover the given part of the stent on said second surface.

Inventive concept 18. The method of Inventive concept 16, wherein each strut-segment within the given area has a transverse cross-section defining a rounded shape, and the respective transverse portion comprises at least 40% of the respective perimeter of each strut-segment.

Inventive concept 19. The method of Inventive concept 16, wherein each strut-segment within the given area has a transverse cross-section defining a prismatic shape, and the respective transverse portion comprises at least 60% of the respective perimeter of each strut-segment.

Inventive concept 20. The method of Inventive concept 17, wherein each strut-segment within the given area has a transverse cross-section defining a rounded shape, and the respective transverse portion comprises at least 80% of the respective perimeter of each strut-segment.

Inventive concept 21. The method of Inventive concept 17, wherein each strut-segment within the given area has a transverse cross-section defining a prismatic shape, and the respective transverse portion comprises at least 90% of the respective perimeter of each strut-segment.

Inventive concept 22. The method of any one of Inventive concepts 16 to 21, wherein at least one respective transverse portion is discontinuous.

Inventive concept 23. The method of any one of Inventive concepts 17 to 22, wherein all of the respective transverse portions are discontinuous.

Inventive concept 24. The method of any one of Inventive concepts 16 to 23, wherein the coating includes applying a coupling agent between the polymer binder and the respective transverse portions to form a covalent bond therebetween.

Inventive concept 25. The method of any one of Inventive concepts 17 to 24, wherein the further coating includes applying a coupling agent between the polymer binder and the respective transverse portions to form a covalent bond therebetween.

Inventive concept 26. The method of any one of Inventive concepts 16 to 25, wherein the engaging includes applying a coupling agent to mediate between the polymer binder and the fabric material to form a covalent bond therebetween.

Inventive concept 27. The method of any one of Inventive concepts 17 to 26, wherein the additionally engaging includes applying a coupling agent to mediate between the polymer binder and the fabric material to form a covalent bond therebetween.

Inventive concept 28. The method of any one of Inventive concepts 17 to 27, wherein the fabric material comprises a substantially impermeable layer and at least one permeable layer.

Inventive concept 29. The method of Inventive concept 28, wherein at least one of the engaging and the further engaging includes causing a material of the substantially impermeable layer to enter spaces of the at least one permeable layer.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

1. A stent assembly comprising:
a. a stent formed by a network of strut-segments and comprising an internal surface and an external surface, each strut-segment having a transverse cross-section defining a respective shape;
b. a fabric material covering a given part of the stent on a first surface selected from the internal surface and the external surface; and
c. a polymer binder,
wherein
i. the covering by the fabric material is such that the fabric material is bonded to a respective transverse portion of a perimeter of each strut-segment within the given part by the polymer binder, and
ii. the respective transverse portion comprises at least 30%, or at least 35%, or at least 40%, or at least 45%, or at least 50%, of the respective perimeter of each strut-segment having a transverse cross-section defining a rounded shape, and at least 55%, or at least 60%, or at least 65%, or at least 70%, of the respective perimeter of each strut-segment having a transverse cross-section defining a prismatic shape.

2. The stent assembly of claim 1, wherein (i) the fabric material additionally covers the given part on a second surface, such that both the internal surface and the external surface of the given part are covered, and (ii) the respective transverse portion comprises at least 70%, or at least 75%, or at least 80%, or at least 85%, or at least 90%, of the respective perimeter of each strut-segment having a transverse cross-section defining a rounded shape, and at least 80%, or at least 85%, or at least 90%, or at least 95%, of the respective perimeter of each strut-segment having a transverse cross-section defining a prismatic shape.

3. The stent assembly of claim 1, wherein each strut-segment within the given area has a transverse cross-section defining a rounded shape, and the respective transverse portion comprises at least 40% of the respective perimeter of each strut-segment.

4. The stent assembly of claim 1, wherein each strut-segment within the given area has a transverse cross-section defining a prismatic shape, and the respective transverse portion comprises at least 60% of the respective perimeter of each strut-segment.

5. The stent assembly of claim 2, wherein each strut-segment within the given area has a transverse cross-section defining a rounded shape, and the respective transverse portion comprises at least 80% of the respective perimeter of each strut-segment.

6. The stent assembly of claim 2, wherein each strut-segment within the given area has a transverse cross-section defining a prismatic shape, and the respective transverse portion comprises at least 90% of the respective perimeter of each strut-segment.

7. The stent assembly of claim 1, wherein at least one respective transverse portion is discontinuous.

8. The stent assembly of claim 2, wherein all of the respective transverse portions are discontinuous.

9. The stent assembly of claim 1, additionally comprising a coupling agent mediating between the polymer binder and the respective transverse portions.

10. The stent assembly of claim 9, wherein the coupling agent is effective to create a covalent bond between the polymer binder and the respective transverse portions.

11. The stent assembly of claim 1, additionally comprising a coupling agent mediating between the polymer binder and the fabric material.

12. The stent assembly of claim 11, wherein the coupling agent is effective to create a covalent bond between the polymer binder and the fabric material.

13. The stent assembly of claim 1, wherein a ratio of a thickness of the strut-segments to a thickness of the polymer binder is at least 1.5.

14. The stent assembly of claim 1, wherein a ratio of a thickness of the strut-segments to a thickness of the polymer binder is at least 2.

15. A method for assembling a stent assembly, the method comprising:
a. providing a stent that comprises an internal surface and an external surface, and that is formed by a network of strut-segments, each strut-segment having a transverse cross-section defining a respective shape;
b. coating, with a polymer binder, a respective transverse portion of a perimeter of each strut-segment within a given part of the stent on a first surface selected from the internal surface and the external surface, wherein the coated respective transverse portion comprises at least 30%, or at least 35%, or at least 40%, or at least 45%, or at least 50%, of the respective perimeter of each strut-segment having a transverse cross-section defining a rounded shape, and at least 55%, or at least 60%, or at least 65%, or at least 70%, of the respective perimeter of each strut-segment having a transverse cross-section defining a prismatic shape; and
c. engaging a fabric material with the coated respective transverse portions so as to bond the fabric material thereto and thereby cover the given part of the stent on the first surface.

16. The method of claim 15, additionally comprising:
i. further coating, with the polymer binder, the respective transverse portion of the perimeter of each strut-segment within the given part of the stent on a second surface, wherein the respective transverse portion comprises at least 70%, or at least 75%, or at least 80%, or at least 85%, or at least 90%, of the respective perimeter of each strut-segment having a transverse cross-section defining a rounded shape, and at least 80%, or at least 85%, or at least 90%, or at least 95%, of the respective perimeter of each strut-segment having a transverse cross-section defining a prismatic shape, and
ii. additionally engaging a fabric material with the further-coated respective transverse portions so as to bond the fabric material thereto and thereby additionally cover the given part of the stent on said second surface.

17. The method of claim 15, wherein each strut-segment within the given area has a transverse cross-section defining a rounded shape, and the respective transverse portion comprises at least 40% of the respective perimeter of each strut-segment.

18. The method of claim 15, wherein each strut-segment within the given area has a transverse cross-section defining a prismatic shape, and the respective transverse portion comprises at least 60% of the respective perimeter of each strut-segment.

19. The method of claim 16, wherein each strut-segment within the given area has a transverse cross-section defining a rounded shape, and the respective transverse portion comprises at least 80% of the respective perimeter of each strut-segment.

20. The method of claim 16, wherein each strut-segment within the given area has a transverse cross-section defining a prismatic shape, and the respective transverse portion comprises at least 90% of the respective perimeter of each strut-segment.

21-56. (canceled)
